(19) United States
(12) Patent Application Publication
(10) Pub. No.: US 2026/0048998 A1
(43) Pub. Date: Feb. 19, 2026

(54) METAL OXIDE LAYER, METHOD OF PRODUCING THE SAME, AND ORGANIC PHOTOVOLTAIC CELL COMPRISING THE SAME

(71) Applicant: THE HONG KONG POLYTECHNIC UNIVERSITY, Hong Kong (CN)

(72) Inventors: Gang LI, Hong Kong (CN); Jiaming HUANG, Hong Kong (CN)

(21) Appl. No.: 19/300,930

(22) Filed: Aug. 15, 2025

Related U.S. Application Data
(60) Provisional application No. 63/683,463, filed on Aug. 15, 2024.

(51) Int. Cl. C01G 9/02; B82B 3/00; C07C 65/05; C07C 65/21; H01G 9/20

(52) U.S. Cl. CPC C01G 9/02; B82B 3/0009; C07C 65/05; C07C 65/21; H01G 9/20

(57) ABSTRACT

A metal oxide layer comprising a modified metal oxide nanoparticle, wherein the modified metal oxide nanoparticle comprises an organic acid metal salt on the surface of a metal oxide nanoparticle, and the organic acid metal salt has Formula 1, wherein m is a whole number selected from 0-2; n is a whole number selected from 0-12; X is —O— or a bond; R¹ for each instance is independently H, OH, alkyl or cycloalkyl; R² for each instance is independently hydrogen or alkyl; or two instances of —CR²— taken together form a double bond; and [symbol] represents a metal counterion. An organic photovoltaic cell comprising the metal oxide layer can achieve improved PCE and stability.

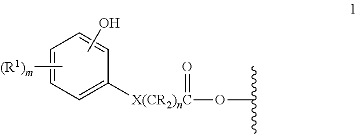

### CROSS-REFERENCE TO RELATED APPLICATIONS

The present application claims priority from U.S. Provisional Patent Application No. 63/683,463, filed on Aug. 15, 2024, which is hereby incorporated by reference in its entirety.

### TECHNICAL FIELD

The present disclosure relates to a metal oxide layer, a method of producing the same and an organic photovoltaic cell comprising the metal oxide layer.

### BACKGROUND

Organic photovoltaics (OPV) have shown great potential for energy conversion in specific applications, such as transparent and wearable devices, due to properties like low-cost, light-weight, non-toxic, transparency and flexibility. Currently, OPVs with the highest Power Conversion Efficiency (PCE) generally rely on (p-i-n) structures based on the poly(3,4-ethylenedioxythiophene):poly(styrene sulfonate) “PEDOT:PSS” hole transport layer, which poses huge challenges to ambient stability due to deliquescence. For the successful commercialization of OPV, addressing the lifetime-related issues in highly efficient OPV from both material- and device-level are crucial.

Interlayer plays a vital role in both efficiency and stability of OPV. Inverted (n-i-p) OPV have long been shown to be more promising in terms of device stability, especially benchmark metal oxide (e.g., ZnO) as bottom electron transport layers (ETL). However, for the state-of-the-art OPV with non-fullerene acceptors, the inverted devices have not realized the high expectations—they tend to exhibit relatively lower power conversion efficiency (PCE) (reported 18.75 and certified 16.76%, see Table S6 in FIG. 40) compared to regular devices, and the stability is also unsatisfactory. This performance discrepancy is connected to the abundance of surface oxygen vacancies (Vₒ⁺) on the metal oxide, leading to trap-assisted charge carrier recombination. In addition, the adsorption of hydroxyl ions, O₂ and H₂O molecules on Vₒ⁺ result in unfavorable light-soaking problem.

There is therefore a need to improve PCE and stability of organic photovoltaic devices. The subject matter described herein addresses at least some of these unmet needs.

### SUMMARY

The present disclosure provides strategies for improving PCE and stability of organic photovoltaic devices through effective passivation of surface oxygen vacancy and reactive radical capture so as to enhance the performance in the photovoltaic devices.

In a first aspect, provided herein is a metal oxide layer comprising a modified metal oxide nanoparticle, wherein the modified metal oxide nanoparticle comprises an organic acid metal salt on the surface of a metal oxide nanoparticle, and the organic acid metal salt has Formula 1:

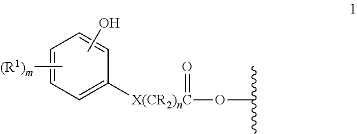

wherein
m is a whole number selected from 0-2;
n is a whole number selected from 0-12;
X is —O— or a bond;
R¹ for each instance is independently H, OH, alkyl or cycloalkyl;
R² for each instance is independently hydrogen or alkyl; or two instances of —CR²— taken together form a double bond; and
[symbol] represents a metal counterion.

In certain embodiments, the metal oxide nanoparticle comprises an n-type metal oxide.

In certain embodiments, the metal oxide nanoparticle comprises ZnO, SnO₂, TiO₂, In₂O₃, NiO, VOₓ, WO₃, or MoO₃.

In certain embodiments, R¹ for each instance is independently branched alkyl.

In certain embodiments, X is a bond, R¹ for each instance is branched alkyl, R² for each instance is hydrogen.

In certain embodiments, m is a whole number selected from 1-2, and n is a whole number selected from 1-5.

In certain embodiments, X is a bond, R¹ for each instance is OH, R² for each instance is hydrogen or alkyl

In certain embodiments, m is a whole number selected from 1-2, and n is a whole number selected from 0-3.

In certain embodiments, the metal oxide nanoparticle comprises ZnO.

In certain embodiments, the modified metal oxide nanoparticle has an average particle size of 5-10 nm.

In a second aspect, provided herein is a method for producing the metal oxide layer of the first aspect, the method comprising
providing a dispersion of modified metal oxide nanoparticle, and annealing the dispersion to form the metal oxide layer;
wherein the modified metal oxide nanoparticle is prepared by contacting a metal oxide nanoparticle with an organic acid thereby forming an organic acid metal salt distributed on the surface of the metal oxide nanoparticle; and the organic acid is represented by Formula 2:

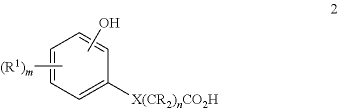

wherein
m is a whole number selected from 0-2;
n is a whole number selected from 0-12;
X is —O— or a bond;
R¹ for each instance is independently H, OH, alkyl or cycloalkyl; and
R² for each instance is independently hydrogen or alkyl; or two instances of —CR²— taken together form a double bond.

In certain embodiments, X is a bond, R¹ for each instance is branched alkyl, R² for each instance is hydrogen.

In certain embodiments, X is a bond, R¹ for each instance is OH, R² for each instance is hydrogen or alkyl.

In certain embodiments, the organic acid is selected from the group consisting of:

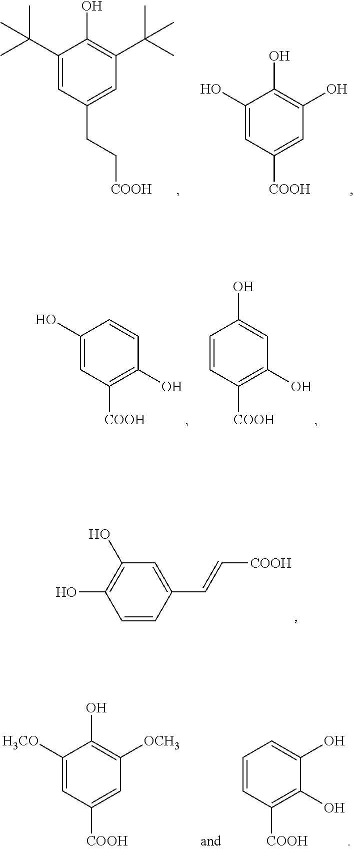

In certain embodiments, the dispersion of modified metal oxide nanoparticle comprises a solvent, and the solvent comprises methanol, isopropanol, butanol, dimethyl formamide (DMF), dimethyl sulfoxide, N-methyl-2-pyrrolidone (NMP), N,N′-dimethylpropyleneurea (DMPU), or mixtures thereof.

In certain embodiments, the metal oxide nanoparticle and the organic acid are contacted at a temperature of 10-50° C.

In a third aspect, provided herein is a coating structure comprising the metal oxide layer of the first aspect and an active layer disposed on a surface of the metal oxide layer.

In certain embodiments, the active layer comprises poly[(2,6-(4,8-bis(5-(2-ethylhexyl-3-fluoro)thiophen-2-yl)-benzo[1,2-b:4,5-b′]dithiophene))-alt-(5,5-(1′,3′-di-2-thienyl-5′,7′-bis(2-ethylhexyl)benzo[1′,2′-c:4′,5‘-c’]dithiophene-4,8-dione)](PM6), 2,2′-[[12,13-Bis(2-butyloctyl)-12,13-dihydro-3,9-dinonylbisthieno[2″,3″:4′,5′]thieno[2′,3′:4,5]pyrrolo[3,2-e:2′,3′-g][2,1,3]benzothiadiazole-2,10-diyl]bis[methylidyne(5,6-chloro-3-oxo-1H-indene-2,1(3H)-diylidene)]]bis[propanedinitrile](BTP-eC9), 2,2′-((2Z,2′Z)-((12,13-bis(2-ethylhexyl)-3,9-(2-butyloctyl)-12,13-dihydro-[1,2,5]thiadiazolo[3,4-e]thieno[2″,3″:4′,5′]thieno[2′,3′:4,5]pyrrolo[3,2-g]thieno[2′,3′:4,5]thieno[3,2-b]indole-2,10-diyl)bis(methanylylidene))bis(5,6-difluoro-3-oxo-2,3-dihydro-1H-indene-2,1-diylidene))dimalononitrile(L8-BO), 2,2′-((2Z,2′Z)-((12,13-bis(2-ethylhexyl)-3,9-diundecyl-12,13-dihydro-[1,2,5]thiadiazolo[3,4-e]thieno[2″,3″:4′,5′]thieno[2′,3′:4,5]pyrrolo[3,2-g]thieno[2′,3′:4,5]thieno[3,2-b]indole-2,10-diyl)bis(methanylylidene))bis(5,6-difluoro-3-oxo-2,3-dihydro-1H-indene-2,1-diylidene))dimalononitrile(Y6), poly[(2,6-(4,8-bis(5-(2-ethylhexyl)-4-fluoro)thiophen-2-yl)-benzo[1,2-b:4,5-b′]dithiophene)-alt-5,5′-(5,8-bis(4-(2-butyloctyl)thiophen-2-yl)dithieno[3′,2′:3,4;2″,3″:5,6]benzo[1,2-c][1,2,5]thiadiazole)](D18), poly[(2,6-(4,8-bis(5-(2-ethylhexyl)-4-chlorothiophen-2-yl)-benzo[1,2-b:4,5-b′]dithiophene))-alt-5,5′-(5,8-bis(4-(2-butyloctyl)thiophen-2-yl)dithieno[3′,2′:3,4;2″,3″:5,6]benzo[1,2-c][1,2,5]thiadiazole)](D18-C1), poly[[12,13-bis(2-octyldodecyl)-12,13-dihydro-3,9-diundecylbisthieno[2″,3″:4′,5′]thieno[2′,3′:4,5]pyrrolo[3,2-e:2′,3′-g][2,1,3]benzothiadiazole-2,10-diyl]methylidyne[1-(dicyanomethylene)-1,3-dihydro-3-oxo-2H-inden-yl-2-ylidene]-2,5-thiophenediyl[1-(dicyanomethylene)-1,3-dihydro-3-oxo-2H-inden-yl-2-ylidene]methylidyne](PY-IT), poly[[4,8-bis[5-(2-ethylhexyl)-2-thienyl]benzo[1,2-b:4,5-b′]dithiophene-2,6-diyl]-2,5-thiophenediyl[5,7-bis(2-ethylhexyl)-4,8-dioxo-4H,8H-benzo[1,2-c:4,5-c′]dithiophene-1,3-diyl]](PBDBD-T) or mixtures thereof.

In a fourth aspect, provided herein is an organic photoelectric cell comprising the metal oxide layer of the first aspect.

In certain embodiments, the organic photoelectric cell has an inverted structure.

### BRIEF DESCRIPTION OF THE DRAWINGS

The foregoing aspects and many of the attendant advantages of this invention will become more readily appreciated and understood by reference to the following detailed description, when taken in conjunction with the accompanying drawing.

FIG. 1 depicts characterization of BHT@ZnO. (a) Synthesis route of BHT@ZnO; (b) TEM images of BHT@ZnO; (c) FT-IR; (d) XPS O signals; (e) XPS Zn signals; (f) PL spectra; (g) EPR; (h) UPS spectra of ZnO and BHT@ZnO.

FIG. 2 depicts device performance and stability of the devices based on ZnO and BHT@ZnO ETL and PM6: BTP-eC9 active layer. (a) Device structure; (b) J-V curves; (c) EQE spectra; (d) Ambient storage stability and light soaking effect after storage; (e) J-V curves variation during light soaking; (f) Light-dependent VOC variation of fresh and aged devices; MPP tracking of (g) unencapsulated device and (h) encapsulated device in ambient condition.

FIG. 3 depicts morphology evolution of the active layer before and after aging at UV illumination based on ZnO and BHT@ZnO ETL. (a) 2D GIWAXS patterns; (b) 1D GIWAXS profiles; (c) Calculated CCL values; (e) Height images measured from AFM and (f) phase images measured from AFM.

FIG. 4 depicts absorption decay of PM6:BTP-eC9 active layer system under different aging conditions. The active layer coated on glass, ZnO and BHT@ZnO under UV illumination in (a-c) N₂ (O₂ content<10 ppm, H₂O content<10 ppm); (e-g) Dry air (O₂ content≈19%, relative humidity≈1%); (i-k) Ambient (O₂ content≈19%, relative humidity≈60-70%); (d,h,f) Summary of the absorption peak intensity for PM6 and BTP-eC9.

FIG. 5 depicts mechanism investigation of materials deterioration. EPR spectra of the DMPO extracted from BHT@ZnO and ZnO films (a) in UV/N₂ and (b) in UV/air; MALDI-TOF-TOF-MS spectra of (c) 3BDDBDT and (d) 3BDTBDD after aging at UV/air for 168 h; (e) Fitted peaks of PM6 absorption spectra aging under UV/air with different time. Dash lines were obtained from experiment, and colored area were fitted peaks: pink area indicated strong aggregated (ordered packing) phase, blue area indicated amorphous phase; (f) Dihedral angle between BDT (BDT1O, DBT2O) and BDD unit extracted from DFE simulations.

FIG. 6 depicts schematic diagram of the degradation behavior of active layers on ZnO and the working mechanism of BHT@ZnO in improving operational stability of OPV.

FIG. 7 depicts device performance of inverted OPV. J-V curves of OPV devices based on (a) PM6:Y6, (b) PM6:L8-BO, and (c) PM6:BTP-eC9:o-BTP-eC9 systems; (d) Lifetime of inverted OPV based on PM6:BTP-eC9:o-BTP-eC9 active layer and BHT@ZnO ETL under MPP tracking; (e) Statistical figure of PCE vs. Tso of OPV.

FIG. 8 depicts XRD patterns of BHT@ZnO and ZnO.

FIG. 9 depicts (a) Absorption spectrum and (b) Tauc plot of ZnO and BHT@ZnO.

FIG. 10 depicts DFT simulation of the BHT acid. (a) HOMO and LUMO distribution of the BHT-acid; (b) dipole moment of BHT molecule.

FIG. 11 depicts AFM images of (a) ZnO and (b) BHT@ZnO nanoparticles coated on ITO/glass.

FIG. 12 depicts contact angle measurement of the different films.

FIG. 13 depicts energy diagram of the inverted devices.

FIG. 14 depicts chemical structure of the active layer materials.

FIG. 15 depicts (a) Dark current density versus voltage and (b) photogenerated current density versus effective voltage of the devices based on ZnO and BHT@ZnO.

FIG. 16 depicts (a) Transient photovoltage and (b) Transient photocurrent of the devices based on ZnO and BHT@ZnO ETL.

FIG. 17 depicts EIS and equivalent-circuit model based on ZnO and BHT@ZnO devices under dark and reverse bias equal to VOC.

FIG. 18 depicts photovoltaic parameters variations after light illumination of ambient-aged ZnO-based device.

FIG. 19 depicts photovoltaic parameters variations after light illumination of ambient-aged BHT@ZnO-based device.

FIG. 20 depicts detailed parameter (light-soaking recovered) of ZnO and BHT@ZnO-based devices aging at ambient condition.

FIG. 21 depicts XPS images of fresh and aged devices based on (a) ZnO and (b) BHT@ZnO.

FIG. 22 depicts detailed parameter of ZnO and BHT@ZnO-based unencapsulated devices aging at white LED (100 mW·cm⁻²) illumination.

FIG. 23 depicts detailed parameter of ZnO and BHT@ZnO-based encapsulated devices aging at white LED (100 mW cm⁻²) illumination.

FIG. 24 depicts detailed parameter of ZnO and BHT@ZnO-based unencapsulated devices aging at UV (10 mW cm⁻²) illumination in glove box.

FIG. 25 depicts detailed parameter of ZnO and BHT@ZnO-based unencapsulated devices aging at MPP.

FIG. 26 depicts detailed parameter of ZnO and BHT@ZnO-based encapsulated devices based on PM6:BTP-eC9 aging at MPP condition.

FIG. 27 depicts (a) Absorption decay of PM6 neat films (b-e) Absorption decay of BTP-eC9 neat films aging under UV illumination in air with different conditions; (f) Normalized intensity variation of BTP-eC9 peaks.

FIG. 28 depicts EPR spectra of ZnO sample in (a) N₂ under dark condition and (b) ambient under dark condition.

FIG. 29 depicts EPR spectra of the Fenton's reagent with DMPO with or without BHT at different reaction time. (a-b) Fenton's reagent in H₂O with reaction time of 5 and 30 min; (c-d) Fenton's reagent in CH₃OH with reaction time of 5 and 30 min.

FIG. 30 depicts chemical structures and absorption spectra of oligomers (a) 3BDTBDD and (b) 3BDTBDD.

FIG. 31 depicts chemical structures, DFT simulated geometry structures from front view and top view of (a, d, g) BDT-BDD, (b, e, h) BDT1O-BDD, and (c, f, i) BDT20-BDD.

FIG. 32 depicts certificated report from Enli Tech Optoelectronic Calibration Lab (ISO/IEC 17025:2017 accredited Calibration Lab).

FIG. 33 depicts detailed parameter of ZnO and BHT@ZnO-based encapsulated devices based on PM6:BTP-eC9 aging at MPP condition.

FIG. 34 depicts (a) the molecular structure of gallic acid; (b) photographs of ZnO and GA@ZnO dispersed in methanol; (c) photovoltaic performance of inverted OPV based on ZnO and GA@ZnO ETL; and (d) device stability under MPPT.

FIG. 35 depicts Table S1 showing the stoichiometric ratio of XPS peaks.

FIG. 36 depicts Table S2 showing surface tension of different thin films.

FIG. 37 depicts Table S3 showing detailed parameters of the devices based on BHT@ZnO as ETL and PM6:BTP-eC9 as the active layer.

FIG. 38 depicts Table S4 showing the parameters extracted from out-of-plane peaks in GIWAXS characterization.

FIG. 39 depicts Table S5 showing the parameters extracted absorption of pure PM6.

FIG. 40 depicts Table S6 showing literature summary of OPV with Tso lifetime in recent 5 years.

### DETAILED DESCRIPTION

### Definitions

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

The terms “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

As used herein, “alkyl” refers to a straight-chain or branched saturated hydrocarbon group. Examples of alkyl groups include methyl-, ethyl-, propyl (e.g., n-propyl and isopropyl), butyl (e.g., n-butyl, iso-butyl, sec-butyl, tert-butyl), pentyl groups (e.g., 1-methylbutyl, 2-methylbutyl, iso-pentyl, tert-pentyl, 1,2-dimethylpropyl, neopentyl, and 1-ethylpropyl), hexyl groups, and the like. In various embodiments, an alkyl group can have 1 to 40 carbon atoms (i.e., C1-40 alkyl group), for example, 1-30 carbon atoms (i.e., C1-30 alkyl group). In certain embodiments, an alkyl group can have 1 to 6 carbon atoms, and can be referred to as a “lower alkyl group.” Examples of lower alkyl groups include methyl, ethyl, propyl (e.g., n-propyl and isopropyl), and butyl groups (e.g., n-butyl, isobutyl, sec-butyl, tert-butyl). In certain embodiments, alkyl groups can be optionally substituted as described herein. An alkyl group is generally not substituted with another alkyl group, an alkenyl group, or an alkynyl group.

As used herein, “cycloalkyl” by itself or as part of another substituent means, unless otherwise stated, a monocyclic hydrocarbon having between 3-12 carbon atoms in the ring system and includes hydrogen, straight chain, branched chain, and/or cyclic substituents. Exemplary cycloalkyls include cyclopropyl, cyclobutyl, cyclopentyl, cyclohexyl, cycloheptyl, and the like.

The term “optionally substituted” refers to a chemical group, such as alkyl, alkoxy, alkenyl cycloalkyl, aryl, and the like, wherein one or more hydrogen may be replaced with a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like.

The processes and compositions of the present disclosure may comprise, consist essentially of, or consist of the components and ingredients of the present disclosure as well as other ingredients described herein. As used herein, “consisting essentially of means that the methods and compositions may include additional steps, components or ingredients, but only if the additional steps, components or ingredients do not materially alter the basic and novel characteristics of the claimed processes and compositions.

Organic photovoltaic (OPV) cells, also known as organic solar cells, are a type of solar cell that converts sunlight into electricity using organic materials such as polymers and small molecules. These materials are carbon-based and can be synthesized in a laboratory, unlike inorganic materials like silicon that require extensive mining and processing. OPV cells work by absorbing photons of light and generating an electrical current through the flow of electrons in the organic material. The cells are typically made up of multiple layers, including a layer of organic material sandwiched between two electrodes. When light is absorbed, it creates a flow of electrons from one electrode to the other, producing a current.

The organic solar cell in a normal structure typically consists of a transparent conductive oxide (TCO) layer, an electron transport layer (ETL), an active layer, a hole transport layer (HTL), and a metal electrode. In an organic solar cell having an inverted structure, the order of the layers is reversed compared to the normal structure, with the TCO layer followed by the HTL, the active layer, the ETL, and the metal electrode.

The present disclosure provides unexpected improvements of the inverted OPV, including enhancing the PCE and stability of an OPV cell, by including radical scavenger modified metal oxide nanoparticles (RS@MO NPs) in the electron transport layer. Specifically, the RS@MO NPs provide effective surface oxygen vacancy passivation and reactive radical capture capability. The radical scavenger passivates the surface Vₒ⁺ and removes the dangling hydroxyl ions in metal oxide NPs, while reducing surface tension and capturing the reactive radicals which may other. In certain embodiments, the inverted OPV based on the modified metal oxide NPs demonstrates record efficiencies of 19.47%. Advantageously, it exhibited outstanding ISOS-D-1 and ISOS-L-1 lifetime with 94.2% PCE retention (with light-soaking-free behavior) after 8904 h of ambient storage without encapsulation and 81.5% PCE retention after 7724 h of maximum power point (MPP) tracking in real-world measurement.

The present disclosure provides a metal oxide layer comprising a modified metal oxide nanoparticle, wherein the modified metal oxide nanoparticle comprises an organic acid metal salt on the surface of a metal oxide nanoparticle, and the organic acid metal salt has Formula 1:

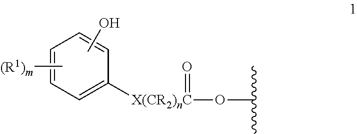

wherein
m is a whole number selected from 0-2;
n is a whole number selected from 0-12;
X is —O— or a bond;
R¹ for each instance is independently H, OH, alkyl or cycloalkyl;
R² for each instance is independently hydrogen or alkyl; or two instances of —CR²— taken together form a double bond; and
[symbol] represents a metal counterion.

In certain embodiments, R¹ for each instance is independently selected from the group consisting of H, —OH, —C₁-C₁₂ alkyl, —C₁-C₉ alkyl, —C₁-C₆ alkyl, —C₁-C₃ alkyl, —C₃-C₇ cycloalkyl, and —C₃-C₅ cycloalkyl.

In certain embodiments, R¹ for each instance is independently branched alkyl. In certain embodiments, R¹ for each instance is independently selected from the group consisting of H, —OH, methyl, ethyl, n-propyl, isopropyl, n-butyl, iso-butyl, tert-butyl, 1-methylbutyl, iso-pentyl, tert-pentyl, 1,2-dimethylpropyl, 1-ethylpropyl, 2-methylpentyl, 2,2-dimethylbutyl, 3,3-dimethylbutyl, 2-methylhexyl, 3-methylhexyl, 2-ethylpentyl 2,2-dimethylpentyl, 2,2-dimethylhexyl, 3,3-dimethylhexyl, 2,2-dimethylheptyl, 2,2-dimethyloctyl, cyclopropyl, cyclobutyl, cyclopentyl, cyclohexyl, and cycloheptyl.

In certain embodiments, R² for each instance is independently selected from the group consisting of H, —C₁-C₉ alkyl, —C₁-C₆ alkyl, and —C₁-C₃ alkyl.

In certain embodiments, R² for each instance is independently selected from the group consisting of methyl, ethyl, n-propyl, isopropyl, n-butyl, iso-butyl and tert-butyl.

In certain embodiments, R² is H and two instances of —CR²— taken together form a double bond.

In certain embodiments, m is a whole number selected from 0, 1 and 2.

In certain embodiments, n is a whole number selected from 0-9, 1-7 or 2-5. In certain embodiments, n is a whole number selected from 0, 1, 2, 3, 4, 5, 6, 7, 8, 9, 10, 11, and 12, or any value or ranges therebetween.

The metal counterion is derived from the metal oxide comprised in the nanoparticle. In certain embodiments, the metal oxide nanoparticle comprises or consists of an n-type metal oxide. In certain embodiments, the metal oxide nanoparticle comprises or consists of ZnO, SnO₂, TiO₂, In₂O₃, NiO, VOₓ, WO₃, or MoO₃.

In certain embodiments, X is a bond, R¹ for each instance is branched alkyl, R² for each instance is hydrogen. In certain embodiments, X is a bond, R¹ for each instance is cycloalkyl alkyl, R² for each instance is hydrogen. In certain embodiments, X is a bond, R¹ for each instance is cycloalkyl alkyl, R² for each instance is hydrogen. In the embodiments, X is a bond, R¹ for each instance is OH, R² for each instance is hydrogen or alkyl.

In certain embodiments, the organic acid metal salt is derived from the organic acid selected from the group consisting of:

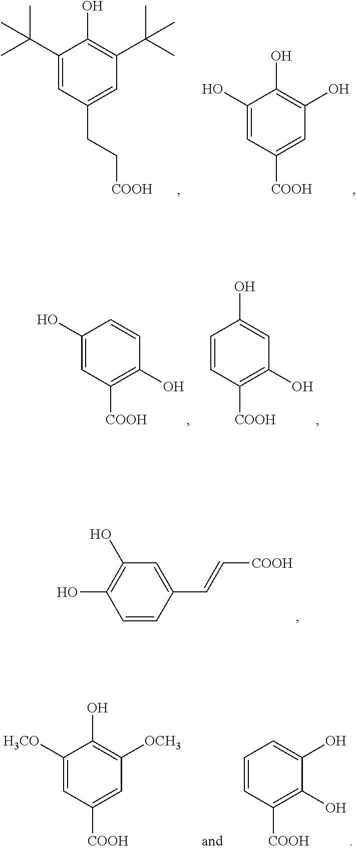

In certain embodiments, the modified metal oxide nanoparticle has high uniformity, and has an average particle size of 5-10 nm. In certain embodiments, the modified metal oxide nanoparticle has an average particle size of 5 nm, 6 nm, 7 nm, 8 nm, 9 nm, 10 nm, or any value or ranges therebetween.

In certain embodiments, a thickness of the metal oxide layer is 10 nm to 200 nm, such as 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or 200 nm, or any value or ranges therebetween.

The present disclosure further provides a method for producing the metal oxide layer as aforementioned, the method comprising:
providing a dispersion of modified metal oxide nanoparticle, and annealing the dispersion to form the metal oxide layer;
wherein the modified metal oxide nanoparticle is prepared by contacting a metal oxide nanoparticle with an organic acid thereby forming an organic acid metal salt distributed on the surface of the metal oxide nanoparticle; and the organic acid is represented by Formula 2:

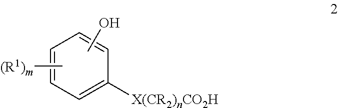

wherein
m is a whole number selected from 0-2;
n is a whole number selected from 0-12;
X is —O— or a bond;
R¹ for each instance is independently H, alkyl or OH; and
R² for each instance is independently hydrogen or alkyl; or two instances of —CR²— taken together form a double bond.

In certain embodiments, the organic acid is selected from the group consisting of:

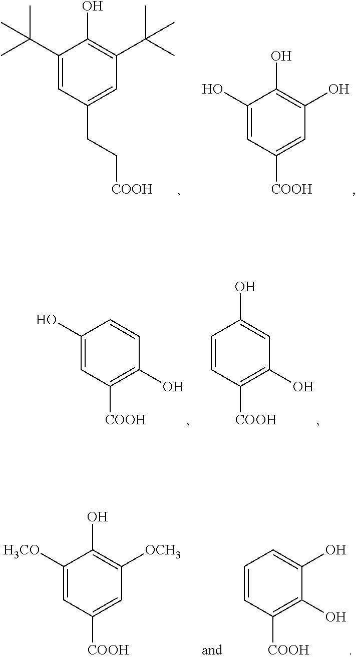

In certain embodiments, the weight ratio of the metal oxide nanoparticle to the organic acid is not particularly limited. For example, the weight ratio of the metal oxide nanoparticle to the organic acid is in the range of 300:1 to 1:10. In certain embodiments, the weight ratio of the metal oxide nanoparticle to the organic acid is 200:1, 150:1, 100:1, 90:1, 80:1, 70:1, 60:1, 50:1, 450:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or any value or ranges therebetween.

In certain embodiments, the metal oxide nanoparticle and the organic acid are contacted at a temperature of 10-50° C., or 20-30° C. In certain embodiments, the metal oxide nanoparticle and the organic acid are contacted at a temperature of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., or any value or ranges therebetween.

In certain embodiments, the metal oxide nanoparticle and the organic acid are contacted to react for a period of 0.5-10 hours. In certain embodiments, the metal oxide nanoparticle and the organic acid are contacted to react for a period of 0.5 hours, 1 hours, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, or any value or ranges therebetween.

In certain embodiments, the metal oxide nanoparticle is produced by a coprecipitation process, a sol-gel method, or a hydrothermal method.

In certain embodiments, the dispersion of modified metal oxide nanoparticle comprises a solvent. In certain embodiments, the solvent comprises methanol, isopropanol, butanol, dimethyl formamide (DMF), dimethyl sulfoxide, N-methyl-2-pyrrolidone (NMP), N,N′-dimethylpropyleneurea (DMPU), or mixtures thereof.

The selection and combination of these solvents can be optimized based on the specific composition and preparation process to achieve the best layer quality and device performance. In certain embodiments, the solvent comprises or consists of methanol.

In certain embodiments, the method further comprises depositing the dispersion of modified metal oxide nanoparticle on a surface to form a wet film; and annealing the wet film to form the metal oxide layer. In some embodiments, the dispersion of modified metal oxide nanoparticle may be deposited by the methods, such as spin coating, blade coating, spray coating, slot-die coating, inkjet printing and vapor deposition. In certain embodiments, the dispersion of modified metal oxide nanoparticle is deposited by spin coating.

Annealing the dispersion of modified metal oxide nanoparticle may significantly impact the layer's quality and the device's performance. Optimal annealing conditions can vary depending on the specific composition and the desired layer properties. In certain embodiments, the dispersion of modified metal oxide nanoparticle or the wet film is subjected to annealing at a temperature of 100-150° C., such as 100° C., 105° C., 110° C., 115° C., 120° C., 125° C., 130° C., 135° C., 140° C., 145° C., or 150° C., or any value or ranges therebetween.

In certain embodiments, the dispersion of modified metal oxide nanoparticle or the wet film is subjected to annealing for a time period of 5-40 min, or 10-30 min. In certain embodiments, the dispersion of modified metal oxide nanoparticle or wet film is subjected to annealing for a time period of 5 min, 10 min, 15 min, 20 min, 25 min, 30 min, 35 min, or 40 min, or any value or ranges therebetween.

The present disclosure further provides a coating structure comprising the metal oxide layer as disclosed herein, and an active layer disposed on a surface of the metal oxide layer.

In certain embodiments, the active layer comprises an organic active material that absorbs solar energy and converts it into free-moving holes and electrons. In certain embodiments, the organic active material comprises poly[(2,6-(4,8-bis(5-(2-ethylhexyl-3-fluoro)thiophen-2-yl)-benzo[1,2-b:4,5-b′]dithiophene))-alt-(5,5-(1′,3′-di-2-thienyl-5′,7′-bis(2-ethylhexyl)benzo[1′,2′-c:4′,5‘-c’]dithiophene-4,8-dione)](PM6), 2,2′-[[12,13-Bis(2-butyloctyl)-12,13-dihydro-3,9-dinonylbisthieno[2″,3″:4′,5′]thieno[2′,3′:4,5]pyrrolo[3,2-e:2′,3′-g][2,1,3]benzothiadiazole-2,10-diyl]bis[methylidyne(5,6-chloro-3-oxo-1H-indene-2,1(3H)-diylidene)]]bis[propanedinitrile](BTP-eC9), 2,2′-((2Z,2′Z)-((12,13-bis(2-ethylhexyl)-3,9-(2-butyloctyl)-12,13-dihydro-[1,2,5]thiadiazolo[3,4-e]thieno[2″,3″:4′,5′]thieno[2′,3′:4,5]pyrrolo[3,2-g]thieno[2′,3′:4,5]thieno[3,2-b]indole-2,10-diyl)bis(methanylylidene))bis(5,6-difluoro-3-oxo-2,3-dihydro-1H-indene-2,1-diylidene))dimalononitrile(L8-BO), 2,2′-((2Z,2′Z)-((12,13-bis(2-ethylhexyl)-3,9-diundecyl-12,13-dihydro-[1,2,5]thiadiazolo[3,4-e]thieno[2″,3″:4′,5′]thieno[2′,3′:4,5]pyrrolo[3,2-g]thieno[2′,3′:4,5]thieno[3,2-b]indole-2,10-diyl)bis(methanylylidene))bis(5,6-difluoro-3-oxo-2,3-dihydro-1H-indene-2,1-diylidene))dimalononitrile(Y6), poly[(2,6-(4,8-bis(5-(2-ethylhexyl)-4-fluoro)thiophen-2-yl)-benzo[1,2-b:4,5-b′]dithiophene)-alt-5,5′-(5,8-bis(4-(2-butyloctyl)thiophen-2-yl)dithieno[3′,2′:3,4;2″,3″:5,6]benzo[1,2-c][1,2,5]thiadiazole)](D18), poly[(2,6-(4,8-bis(5-(2-ethylhexyl)-4-chlorothiophen-2-yl)-benzo[1,2-b:4,5-b′]dithiophene))-alt-5,5′-(5,8-bis(4-(2-butyloctyl)thiophen-2-yl)dithieno[3′,2′:3,4;2″,3″:5,6]benzo[1,2-c][1,2,5]thiadiazole)](D18-C1), poly[[12,13-bis(2-octyldodecyl)-12,13-dihydro-3,9-diundecylbisthieno[2″,3″:4′,5′]thieno[2′,3′:4,5]pyrrolo[3,2-e:2′,3′-g][2,1,3]benzothiadiazole-2,10-diyl]methylidyne[1-(dicyanomethylene)-1,3-dihydro-3-oxo-2H-inden-yl-2-ylidene]-2,5-thiophenediyl[1-(dicyanomethylene)-1,3-dihydro-3-oxo-2H-inden-yl-2-ylidene]methylidyne](PY-IT), poly[[4,8-bis[5-(2-ethylhexyl)-2-thienyl]benzo[1,2-b:4,5-b′]dithiophene-2,6-diyl]-2,5-thiophenediyl[5,7-bis(2-ethylhexyl)-4,8-dioxo-4H,8H-benzo[1,2-c:4,5-c′]dithiophene-1,3-diyl]](PBDBD-T) or mixtures thereof.

The present disclosure further provides an organic photoelectric cell comprising the metal oxide layer.

In certain embodiments, the organic photoelectric cell has an inverted structure. In certain embodiments, the organic photoelectric cell comprises a transparent or translucent conductive substrate as the base or bottom layer, an electron transport layer, an active layer, a hole transport layer and a metal electrode that are deposited sequentially. In certain embodiments, the organic photoelectric cell comprises the metal oxide layer as the electron transport layer.

In certain embodiments, the transparent or translucent conductive substrate may be cleaned in advance by ultrasonication in solvents such as isopropanol, acetone and water. The substrate can also be treated with UV light for surface activation.

The transparent conductive oxide in the conductive substrate is selected from the group consisting of an indium tin oxide (ITO), a zinc oxide, a doped tin oxide, and a doped zinc oxide, such as a fluorine-doped tin oxide (FTO), or an aluminum-doped tin oxide (AZO), etc.

In certain embodiments, the transparent conductive oxide can include 90 wt % to 100 wt % of ITO, FTO, or AZO. In certain embodiments, the transparent conductive oxide can be composed primarily of ITO, FTO, or AZO. In certain embodiments, transparent conductive oxide is ITO.

### EXAMPLES

### Materials

All materials are purchased from commercial suppliers: PM6, BTP-eC9, L8-BO, Y6 (Solarmer Energy Inc.), TCB (Tokyo Chemical Industry Co., Ltd.), Zn(OAc)₂·2H₂O, KOH, BHT-acid (Sigma Aldrich). And all reagents and solvents are used directly without further purification.

### Example 1

### BHT@ZnO Nanoparticles Synthesis

Zn(OAc)₂·2H₂O (2.963 g) was dissolved in methanol (125 ml) at 65° C. for 10 min. After fully dissolved, KOH solution (1.335 g in 65 ml methanol) was added dropwise with 15 min under vigorous stirring. Then the reaction kept at 65° C. for 4 h then stood still for 3 h for fully precipitate. The obtained ZnO nanoparticles were washed twice with methanol. After that, the obtained ZnO nanoparticles was reacted with BHT-acid (0.05 g) at room temperature for 6 h to finish the reaction. Finally, the obtained BHT@ZnO was washed twice with methanol then collected by centrifugation at 16 k rpm and dispersed in methanol to form the dispersion.

### Example 2

### GA@ZnO Nanoparticles Synthesis

The GA@ZnO nanoparticles synthesis is similar to BHT@ZnO. After obtaining ZnO nanoparticles, the GA in methanol was added into ZnO dispersion with similar ratio and react at room temperature for 6 h. And the obtained BHT@ZnO was washed twice with methanol then collected by centrifugation at 16 k rpm and dispersed in methanol to form the dispersion.

### Example 3

### Device Fabrication

First, patterned ITO-coated glass was cleaned by detergent, de-ionized water, acetone, and isopropyl alcohol (IPA) for 40 min under ultrasonication sequentially. The substrate then was dried in N₂ flow and treated with UV ozone for 20 min. The prepared ZnO dispersion, BHT@ZnO NPs dispersion and GA@ZnO dispersion were coated on ITO in glovebox, followed by thermal annealing on a hot plate at 130° C. for 20 min. Then TCB (10 mg/ml) in the PM6: BTP-eC9, PM6:Y6 and PM6:L8-BO (D:A=1:1.2) with the total concentration of 17 mg/ml dissolved in chloroform were coated on the samples, followed by thermal annealing on a hot plate at 100° C. for 10 min. For ternary system, the ratio of PM6:BTP-eC9:o-BTP-eC9 is 1:1.05:0.15 with the total concentration of 16 mg/ml in chloroform with DIB additive (12.5 mg/ml). All the precursor solution would be heated to 60° C. for an hour to fully dissolve the solutes then cooled to room temperature before use. After coating, the active layer experienced a process of thermal annealing at 100° C. for 5 min. 5-nm MoO₃ and 100-nm Ag were then vacuum deposited as the electrode. All devices were tested with a metal mask whose area is ˜6.1 mm². The J-V curves of the OPV were tested by Keithley 2400 source meter and solar simulator (SS-F7-3A, Enli Tech. Co., Ltd., Taiwan) along with AM 1.5 G spectra whose intensity was corrected by a standard silicon solar cell at 100 mW/cm². The EQE was measured by a certified incident photon to electron conversion (IPCE) equipment (QE-R) from Enli Technology Co., Ltd. The lifetime of the device was tested by MPP tracking equipment (Enli Technology Co., Ltd.).

### Method and Characterization

### Contact Angle

The contact angles were tested by a DSA-100 (KRUSS Germany) contact angle meter. The corresponding surface tension was calculated by following Owens-Wendt method:

$${γ}_{L} \times ( 1 + cos θ ) = 2 \times {( {γ}_{L}^{d} \cdot {γ}_{s v}^{d} )}^{1 / 2} + 2 \times {( {γ}_{L}^{p} \cdot {γ}_{s v}^{p} )}^{1 / 2}$$

where and γ_{L} and γₛ are surface tension of the tested liquid and sample, respectively. θ is the contact angle of the sample.

### EPR Measurement

EPR measurements were carried out in ambient condition on ADANI SpinscanX spectrometer, operating at 100 kHz field modulation using the 150 ut modulation amplitude. For the trapping of OH radical, the prepared ETLs were immersed in DMPO anhydrous toluene solution (50 mM) in N₂ under dark or illuminated by AM 1.5G (100 mW·cm⁻²) for 20 min. The sample was drawn by capillary which was furtherly sealed by tweezers after heating by an alcohol lamp. The capillary was placed into the quartz tube, then placed into EPR cavity. For the trapping of O₂ radical, the prepared ETL were immersed in DMPO methanol solution (50 mM) in ambient condition under dark or illuminated by AM 1.5G (100 mW·cm⁻²) for 5 min. Next steps were similar to the trapping of OH radical.

### Preparation of the Fenton's Reagent

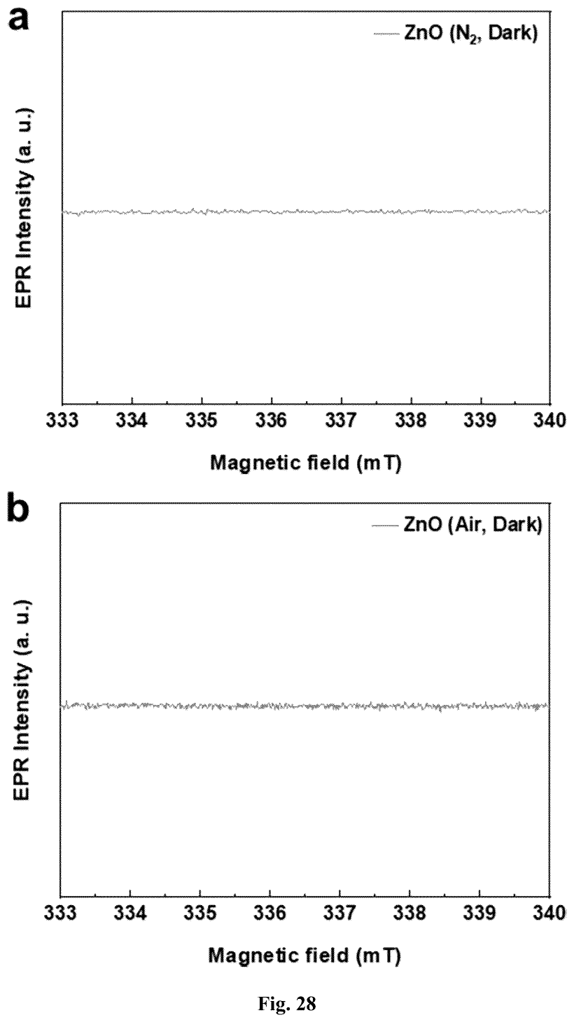

100 μl H₂O₂ and 100 μl FeSO₄ solution (10 mM) were dissolved in 800 μl H₂O to generate OH radical. While 100 μl H₂O₂ and 100 μl FeSO₄ solution (10 mM) were dissolved in 800 μl CH₃OH to generate O₂ radical. 5 μl DMPO was added in mixture solution to measure the radical by EPR. 0.5 mg BHT-acid was added in the mixture for measuring the radical scavenging capability. To improve the catalytic ability, higher concentration of H₂O₂(×20) was used in experiment of active layer bleaching (FIG. 28).

### Other Characterizations

UV-vis absorption spectra and transmittance spectra was carried out by Varian Cary® 300 UV-Vis spectrophotometers (Agilent Technologies). MALDI-TOF/TOF MS was carried out by the Bruker UltrafleXtreme. Atomic force microscopic (AFM) images were acquired using a Bruker Dimension Icon in tapping mode. GIWAXS measurements were carried out with a Xeuss 2.0 SAXS/WAXS laboratory beamline using a Cu X-ray source (8.05 keV, 1.54 Å) and Pilatus3R 300 K detector. The incidence angle is 0.2°. TPV was conducted under 1 sun conditions by illuminating the device with a white light-emitting diode and set at the open-circuit condition. TPC was set to the short-circuit condition in dark. The output signal was collected by keysight oscilloscope.

### Result and Discussion

### 1. Synthesis and Characterization of the BHT@ZnO NPs

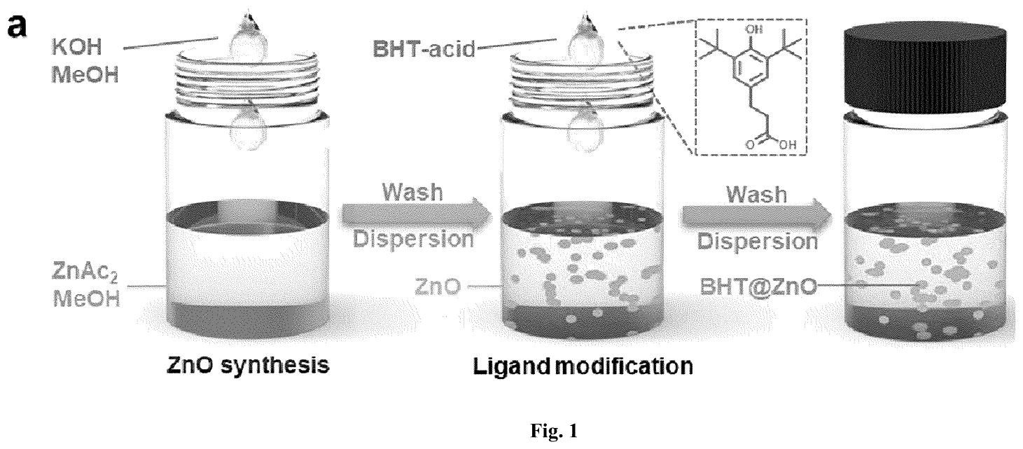

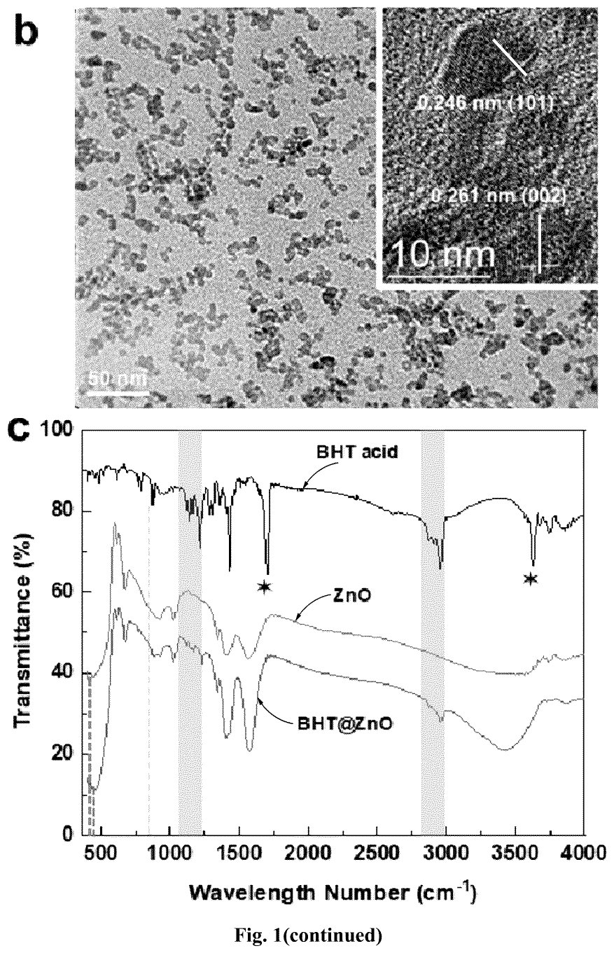

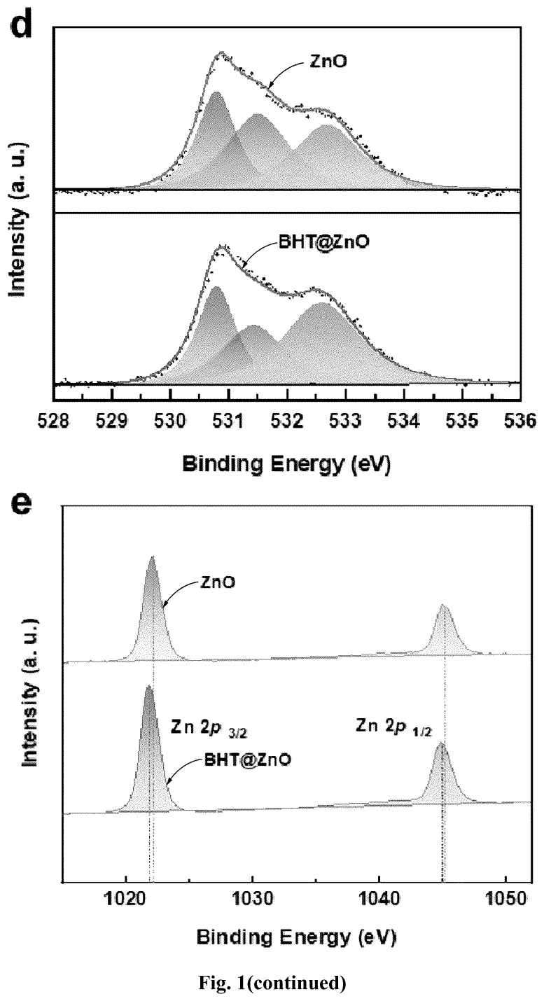

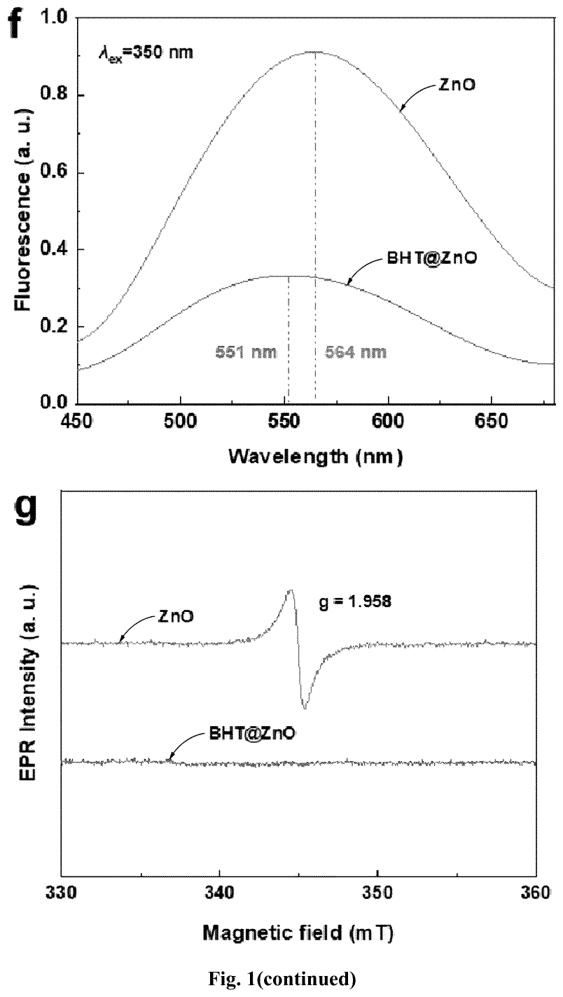

The synthesis of zinc oxide nanoparticles was carried out according to a known method in the literature as a control for comparison, while for the BHT@ZnO NPs, the surface modification of ZnO involved binding it with BHT-acid (FIG. 1a). The carboxylate group enables BHT to anchor on the surface of ZnO. The BHT-liganded ZnO nanoparticles demonstrated high uniformity and an average size of approximately 8 nm based on transmission electron microscopy (TEM) images (FIG. 1b). Additionally, the lattice fringes of the (101) and (002) crystal plane were observed at space distances of approximately 0.246 and 0.261 nm. The formation of BHT@ZnO NPs was verified by X-ray Diffraction (XRD) as evidenced by the characteristic peaks that were assigned to ZnO with a wurtzite structure (FIG. 8).

Fourier transform infrared spectroscopy (FTIR) was used to determine the surface modification of BHT@ZnO (see FIG. 1c). Both BHT-acid and BHT@ZnO NPs exhibit a peak at 881 cm⁻¹, indicating the presence of benzene ring. Peaks in the range of 2800 to 3000 cm⁻¹ and 1100 to 1250 cm⁻¹ (green area) correspond to alkyl groups (tert-butyl and methylene) and phenolic hydroxyl in both BHT-acid and BHT@ZnO, suggesting the presence of BHT moiety in BHT@ZnO NPs. The peaks observed at 3632 and 1708 cm⁻¹ (star marked) indicate free hydroxyl O—H and C═O bond stretching vibration of the carboxylate group in BHT-acid, disappear in BHT@ZnO, indicating the formation of the carboxylate salt (COOZn). Additionally, a high-wavelength-number shift from 428 cm⁻¹ to 454 cm⁻¹ is observed after BHT capping, indicating the formation of new Zn—O bond and the reduction of surface Vₒ⁺.

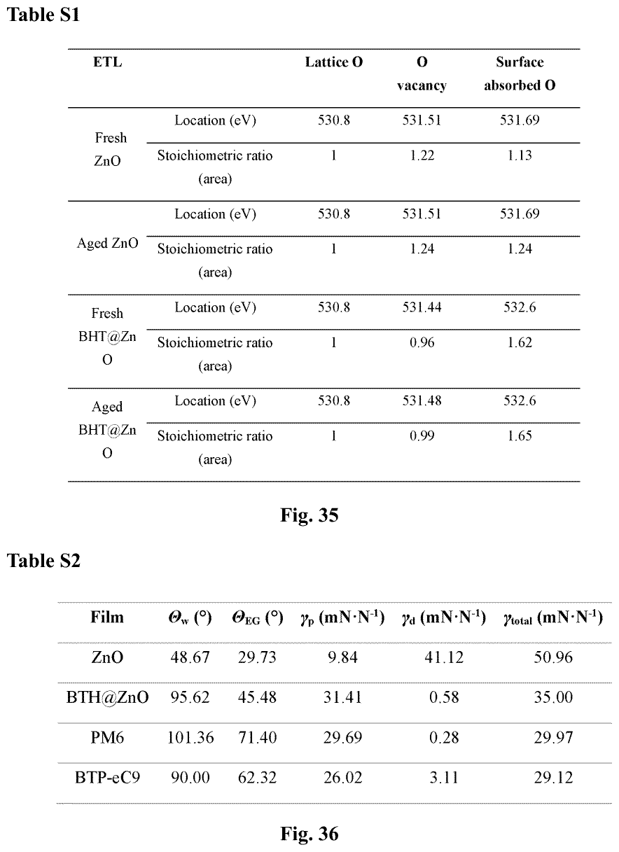

To further identify the surface properties of BHT@ZnO, X-ray photoelectron spectroscopy (XPS) was employed. The O is peak of ZnO NPs was assigned to different binding energies, including 530.8 eV for lattice oxygen (Zn—O), 531.51 eV for Vₒ⁺, and 532.69 eV for surface adsorbed oxygen (FIG. 1d). The stoichiometric ratio of all these peaks is summarized in Table S1 in FIG. 35. The ratio of Vₒ⁺ to lattice oxygen ratio of BHT@ZnO is 0.96, lower than that of ZnO (1.22), which indicates that BHT surface ligand modification passivates the surface defect of ZnO NPs. The higher amount of adsorbed oxygen on the surface of BHT@ZnO is attributed to the presence of oxygen in BHT moiety. Additionally, the Zn 2p peaks of BHT@ZnO NPs showed a lower-binding-energy shift (0.28 eV) than ZnO NPs, which is attributed to the reduced Vₒ⁺ on BHT@ZnO NPs by intensive coordination between COO⁻ and Zn²⁺ (FIG. 1e).

Under 350 nm excitation, ZnO NPs demonstrate strong photoluminescence (PL) emission at 564 nm, which arises from the defect-induced emission related to oxygen-vacancies. However, the photoluminescence emission from BHT@ZnO NPs shows a blue-shift at 551 nm and lower intensity, indicating the passivation of surface oxygen-vacancy-related defects by the carboxylic group in BHT moiety (FIG. 1f). Electron spin resonance (ESR) spectrum was used to further confirm the nature of defects in the nanoparticles, revealing a signal at a g value of 1.958 in ZnO NPs, which is attributed to the presence of Vₒ⁺ defects on the surface. In contrast, EPR signal in BHT@ZnO NPs significantly diminished, indicating the effective passivation by BHT moiety (FIG. 1g).

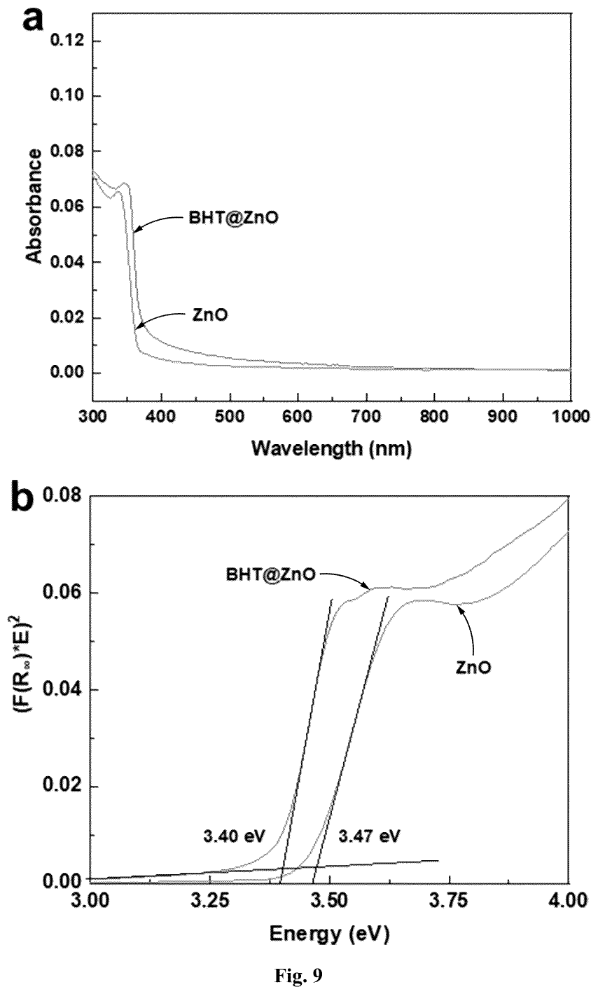

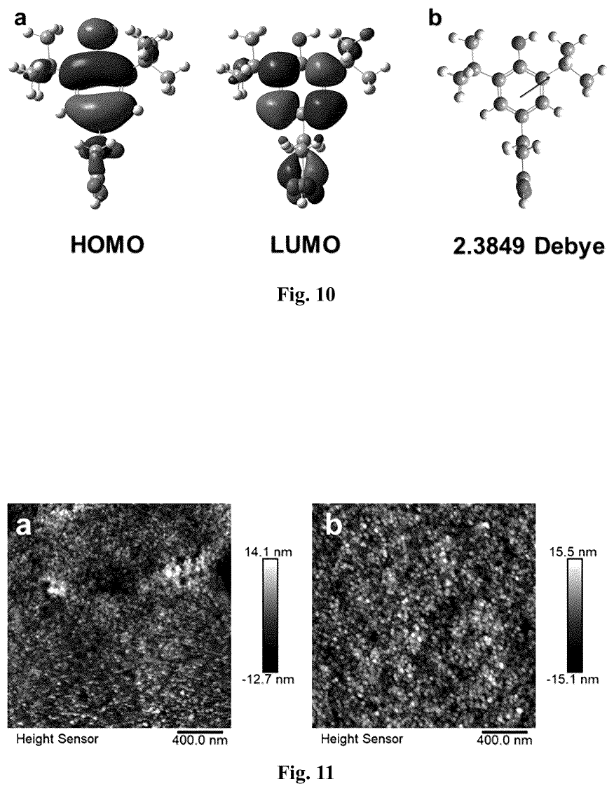

Compared to ZnO, BHT@ZnO thin film displays an approximately 5-nm red-shift in absorbance cutoff according to FIG. 9. The derived Tauc plot indicates that the bandgap of BHT@ZnO has a minor decrease of 3.40 eV in contrast to 3.47 eV of ZnO. Furthermore, we executed ultraviolet photoelectron spectroscopy (UPS) to determine the work function (FIG. 1h). The work function of ZnO was reduced from −4.30 eV to −4.01 eV with the incorporation of the BHT moiety. As indicated by the density functional theory (DFT), this work function variation could be attributed to the dipole of the surface ligands. FIG. 10a shows that the HOMO is predominantly situated in the electron-rich polyphenol portion, while the LUMO is distributed throughout the molecule. As a result, the electron-state density distribution induces a dipole moment of 2.38 Debye, which up-shifts the fermi-level of nanoparticles (FIG. 10b). This work function variation in BHT@ZnO is expected to offer better energy alignment and reduce the Schottky barrier in the ETL/acceptor interface, which is advantageous for charge transport.

The surface properties of the nanoparticles are also studied. Atomic force microscopy (AFM) revealed that both ZnO and BHT@ZnO thin films had smooth and uniform surfaces, with root-mean-square (RMS) roughness values of 2.82 and 3.43 nm, respectively (FIG. 11). Contact angle measurements were taken to calculate the surface tension. Both water and ethylene glycol exhibited higher contact angles on BHT@ZnO film than on ZnO film (FIG. 12). According to Table S2 in FIG. 36, the calculated surface tension of BHT@ZnO film was found to be lower (35.00 mN·N⁻¹) than ZnO films (50.96 mN·N⁻¹). This observation can be attributed to the presence of tert-butyl groups in BHT moiety that prevent water molecule adsorption by BHT@ZnO.

### 2. Photovoltaic Performance

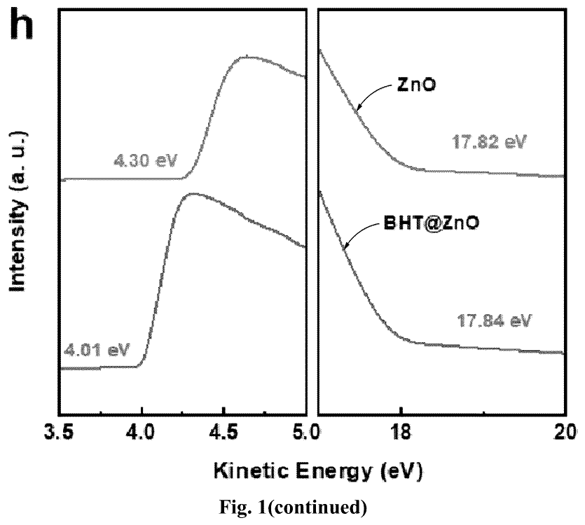

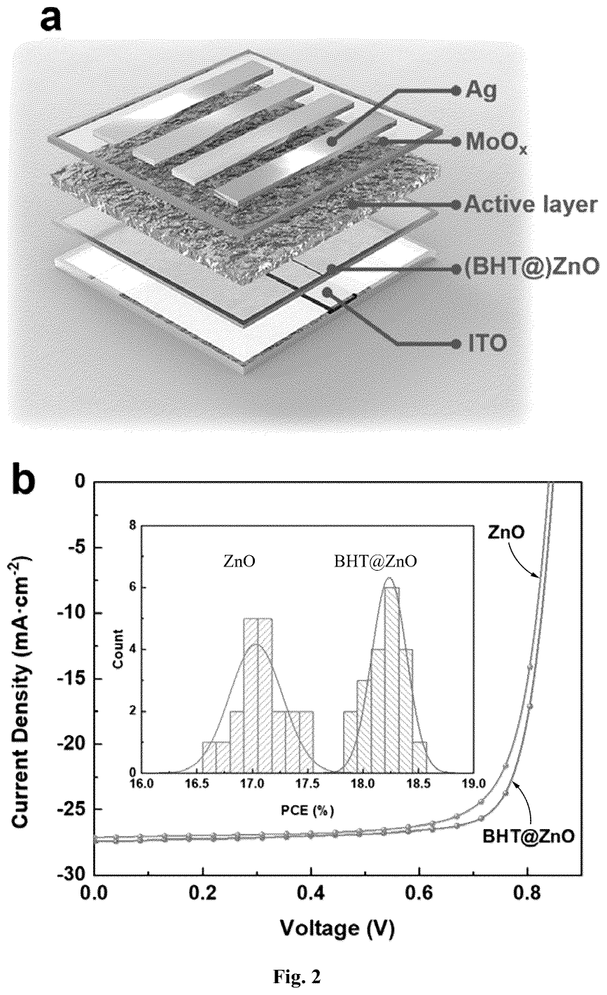

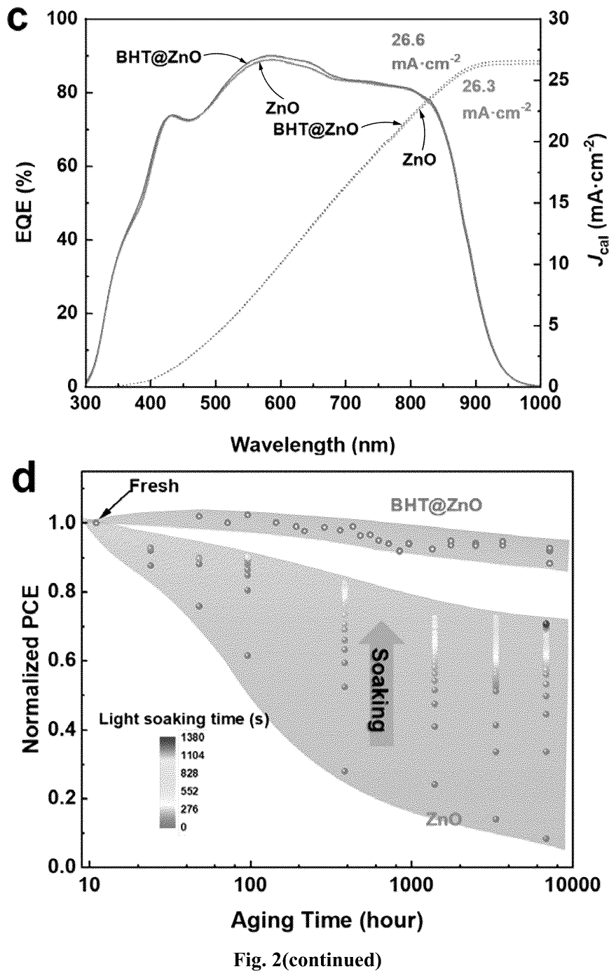

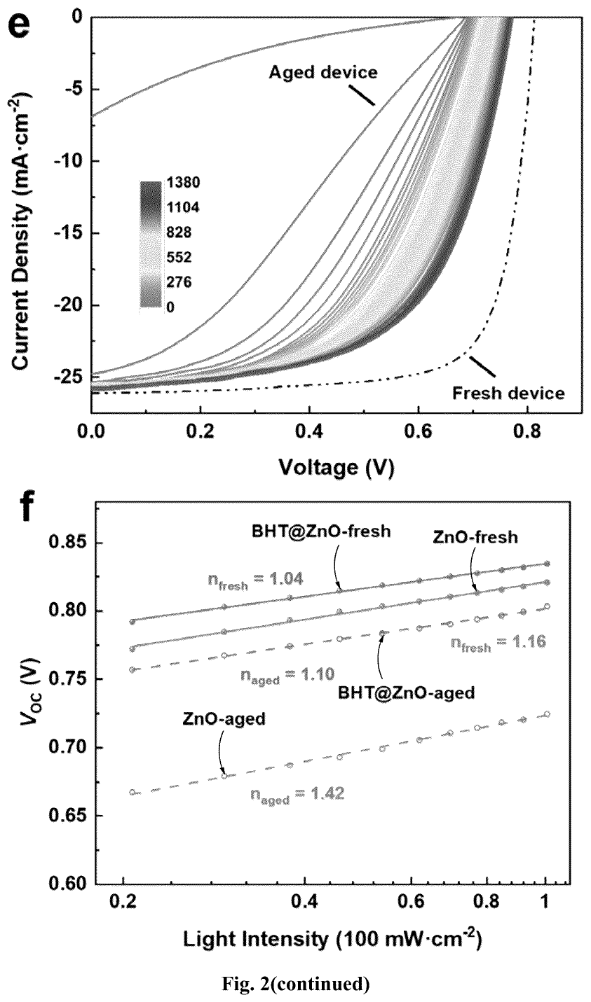

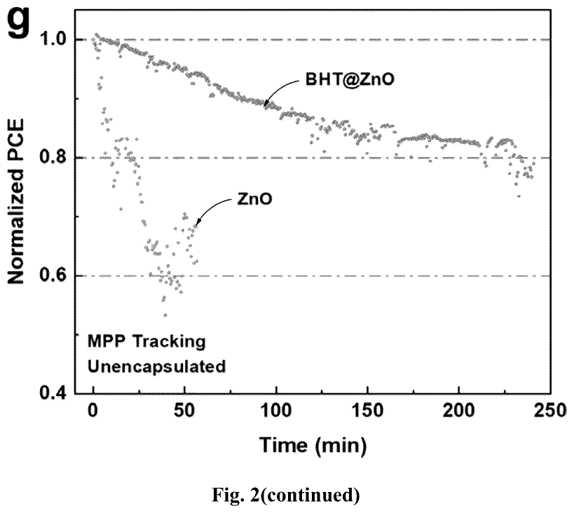

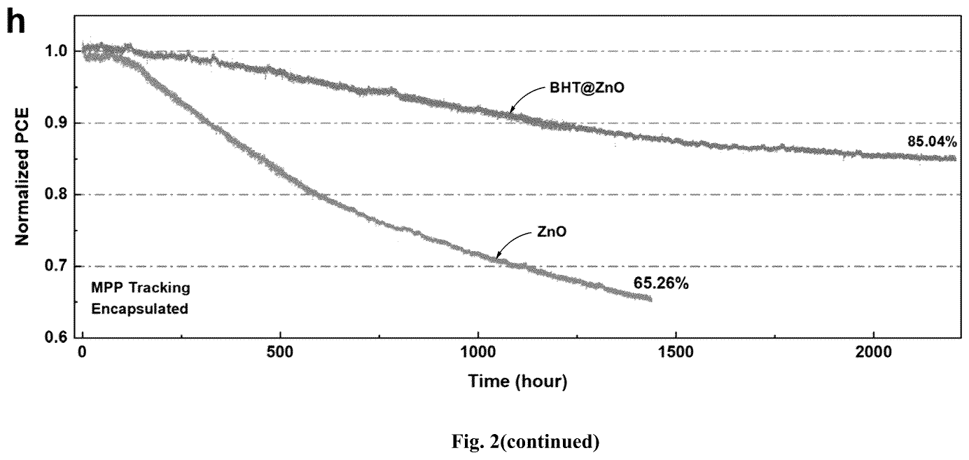

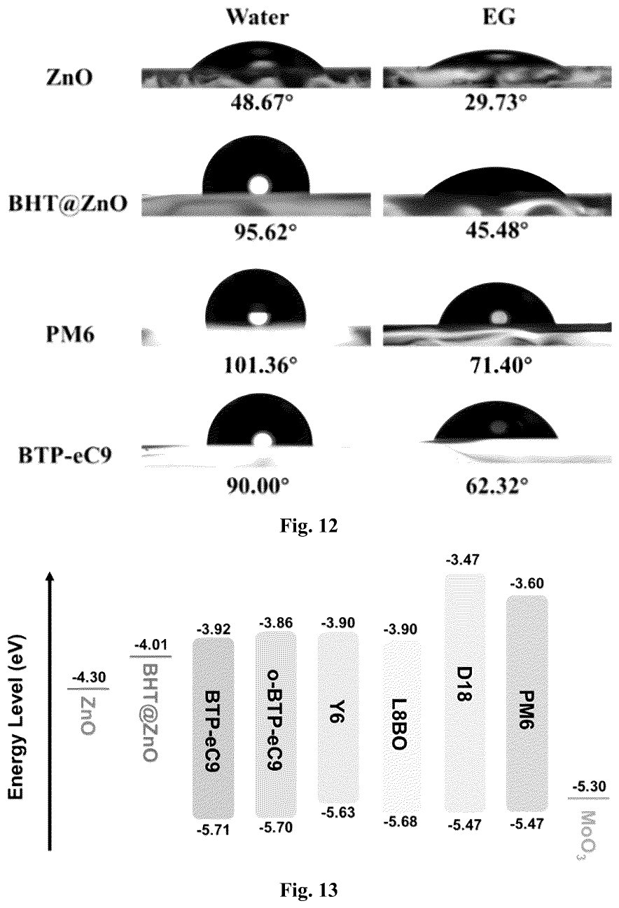

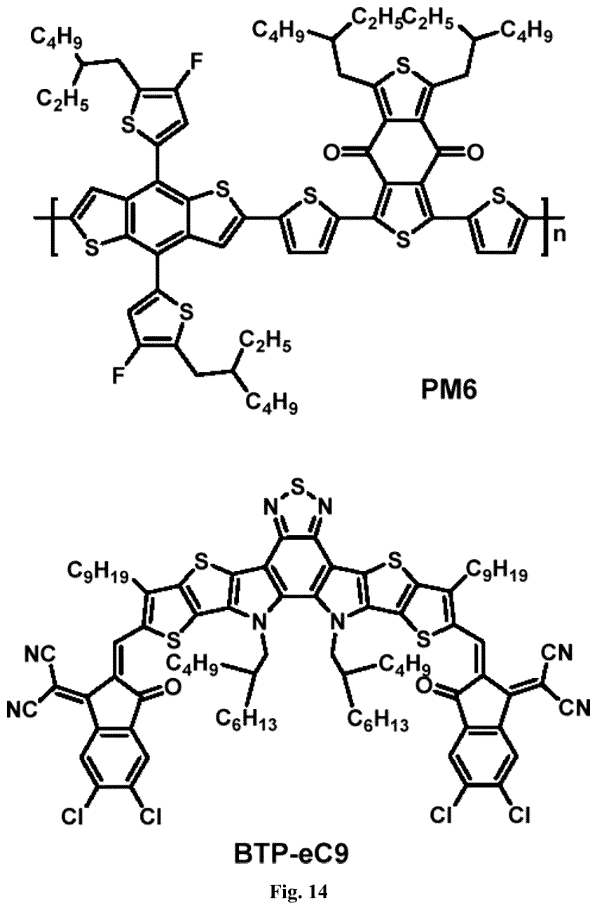

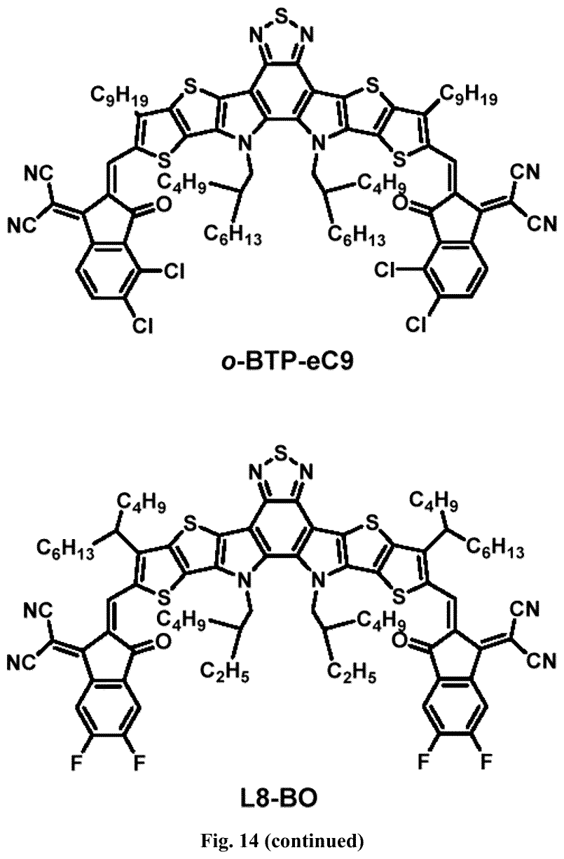

The inverted OPV devices were fabricated with the configuration of ITO/ZnO (BHT@ZnO)/active layer/MoO₃/Ag (FIG. 2a and FIG. 13). The chemical structures of materials in this work are presented in FIG. 14. FIG. 2b shows the current density versus voltage (J-V) characteristics of OPV under simulated AM 1.5G solar illumination with different ETL. Further details on the performance of the devices are outlined in Table 1.

TABLE 1
Photovoltaic parameters of the inverted OPV based on
ZnO and BHT@ZnO with different active layer systems.

| | | | J_{SC} | FF | |
|---|---|---|---|---|---|
| Active layer | ETL | V_{OC} (V) | (mA/cm²) | (%) | PCE (%)^{a} |
| PM6:BTP-eC9 | ZnO | 0.840 | 27.13 | 76.49 | 17.43 (17.03 ± 0.24) |
| | BHT@ZnO | 0.848 | 27.46 | 79.36 | 18.48 (18.23 ± 0.15) |
| PM6:Y6 | ZnO | 0.828 | 26.61 | 74.83 | 16.49 (16.15 ± 0.18) |
| | BHT@ZnO | 0.840 | 26.77 | 77.92 | 17.52 (17.12 ± 0.16) |
| PM6:L8-BO | ZnO | 0.868 | 25.85 | 77.48 | 17.38 (17.01 ± 0.11) |
| | BHT@ZnO | 0.877 | 26.07 | 80.72 | 18.45 (18.06 ± 0.17) |
| PM6:BTP- | ZnO | 0.851 | 27.96 | 77.32 | 18.39 (17.97 ± 0.16) |
| eC9:0-BTP-eC9 | BHT@ZnO | 0.859 | 28.39 | 79.87 | 19.47 (19.13 ± 0.19) |
| | | 0.856 | 28.38 | 78.22 | 18.97^{b} |

^{a}Average PCEs with standard deviation calculated from 20 devices. All devices were tested with a metal mask applied.
^{b}Certificated by Enli Tech Optoelectronic Calibration Lab (ISO/IEC 17025: 2017 accredited Calibration Lab).

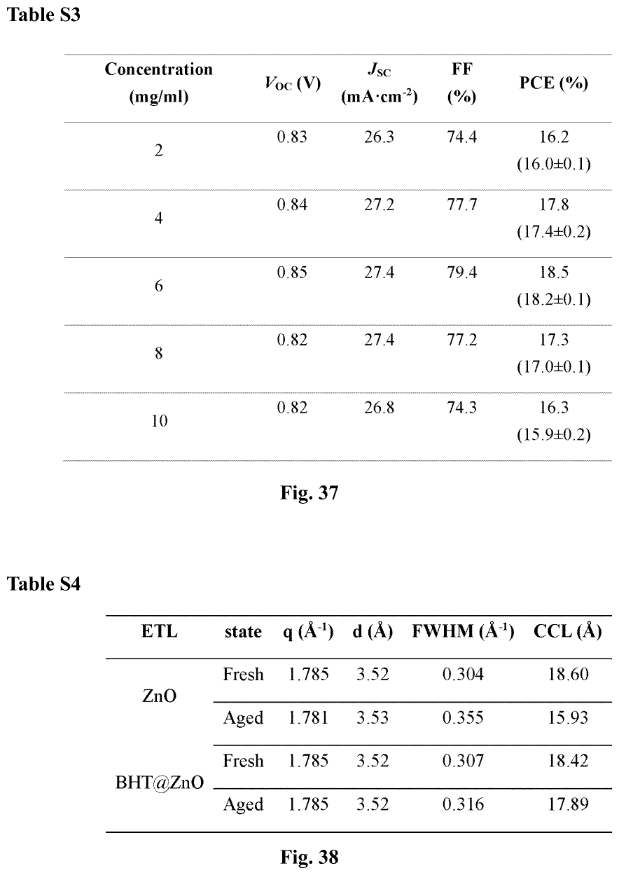

The PCE of device based on ZnO ETL was found to be 17.43% with an open-circuit voltage (V_{OC}) of 0.840 V, a short-circuit current density (J_{SC}) of 27.13 mA·cm⁻², and fill factor (FF) of 76.49%. When BHT@ZnO was used instead of ZnO as ETL, the champion PCE was enhanced to 18.48% with V_{OC} of 0.848 V, J_{SC} of 27.46 mA·cm⁻², and FF of 79.36% after optimization (as shown in Table S3 in FIG. 37). External quantum efficiency (EQE) characterization was used to prove the photon response of the OPV (as shown in FIG. 2c). The calculated integral current densities were found to be 26.6 and 26.3 mA·cm⁻² for BHT@ZnO-based and ZnO-based OPV, respectively, which is consistent with the measured J_{SC}. The BHT@ZnO-based device exhibited improved EQE within the range of 500-900 nm, suggesting better charge transport and collection abilities, even under the same optical transmission of ETL.

Under dark conditions, we investigated the J-V characteristics of devices based on different electron transport layers (ETLs) and found that BHT@ZnO-based device exhibited reduced reversed saturated (leakage) current under reversed bias in comparison to ZnO-based device (FIG. 15a). To further understand the charge transport and collection ability, the relationship between photocurrent density (Jₚₕ) and effective voltage (V_{eff}) was analyzed. As seen in the FIG. 15b, charge dissociation/transport probability (P) increased from 95.70% to 98.36% when modified with BHT moiety, indicating enhanced charge transport of BHT@ZnO.

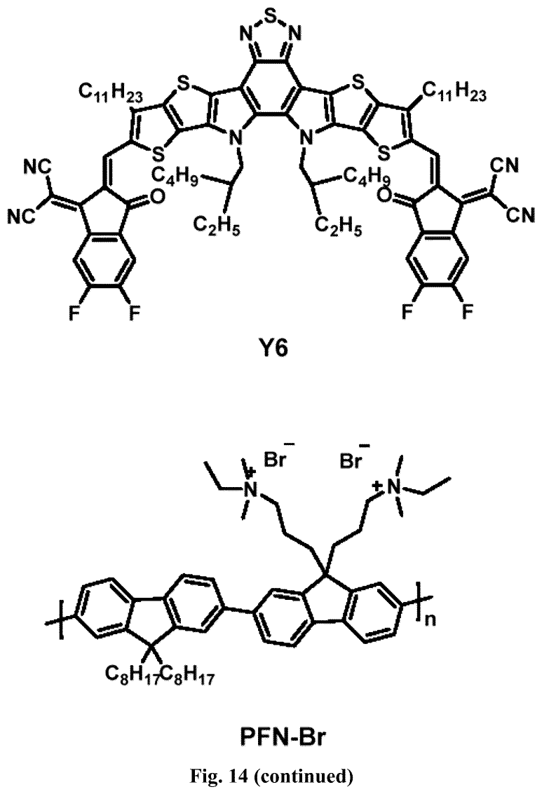

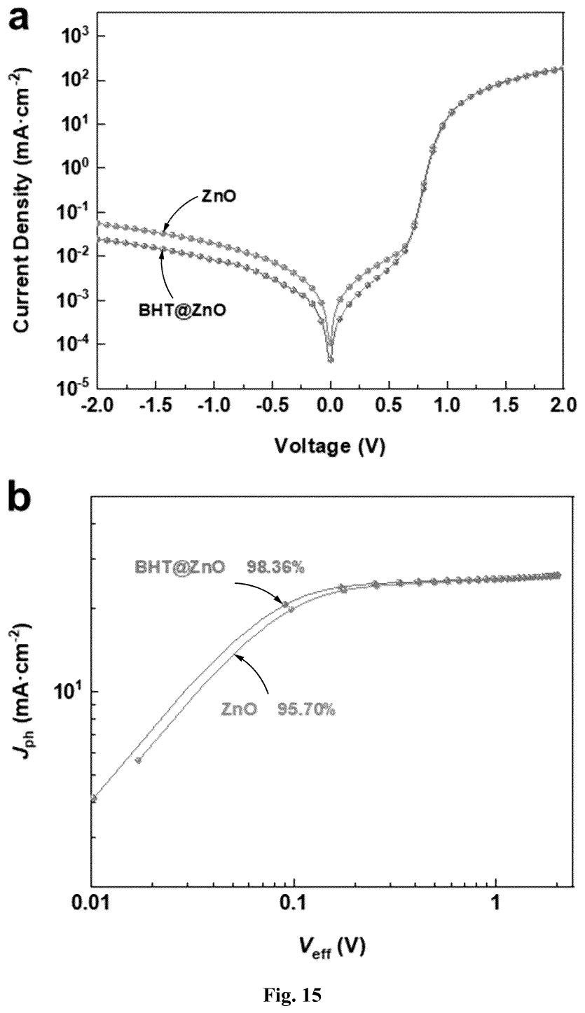

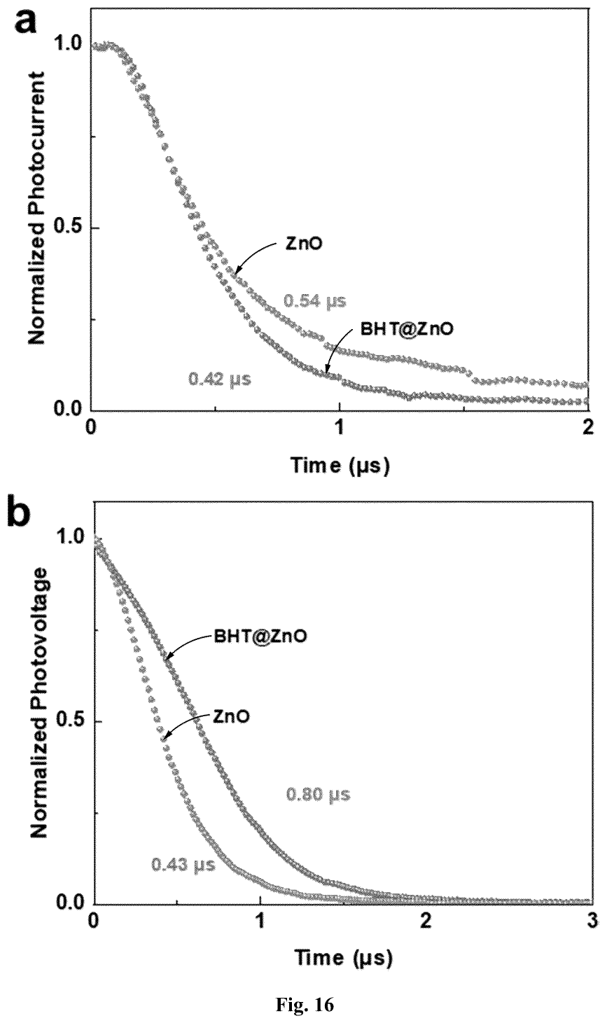

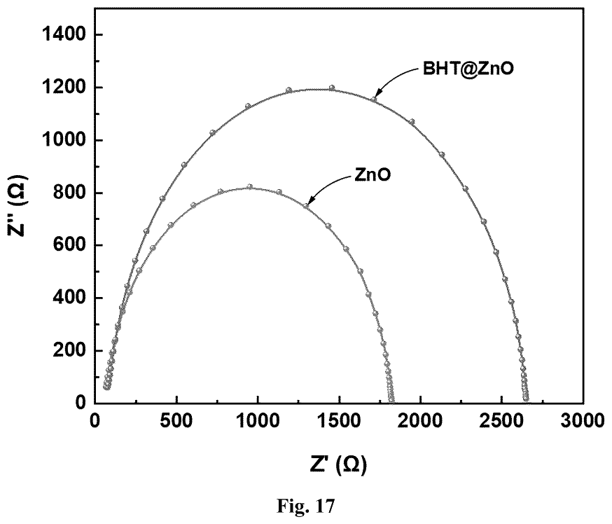

The ideality factor (n) fitting from light-dependent V_{OC} was 1.16 and 1.04 for ZnO and BHT@ZnO, respectively (FIG. 2f). The decreased n value represented suppressed trap-assisted recombination, which is resulted from the reduced surface defects in ETL. To furtherly verify, transient photocurrent (TPC) and transient photovoltage (TPV) techniques were measured to evaluate the recombination (FIG. 16). Compared to ZnO-based device, the BHT@ZnO-based device showed accelerated charge extraction (0.42 s versus 0.54 s) and longer carrier lifetime (0.80 s versus 0.43 s), consistent with the reduced trap-assist recombination. Furthermore, electrochemical impedance spectroscopy (EIS) was conducted with a bias equals to V_{OC} to investigate the interfacial resistance. From the fitted equivalent-circuit model, the ZnO-based device had series resistance (Rₛ) and recombination resistance (R_{rec}) of 62.57 Q and 1762 Q, respectively, while the BHT@ZnO-based device had Rₛ and R_{rec} of 68.26 Q and 2605 Q, respectively (FIG. 17). This reflects the reduced trap-assisted recombination in the BHT@ZnO devices, which is attributed to the surface passivation of ZnO nanoparticles by BHT.

### 3. Device Stability

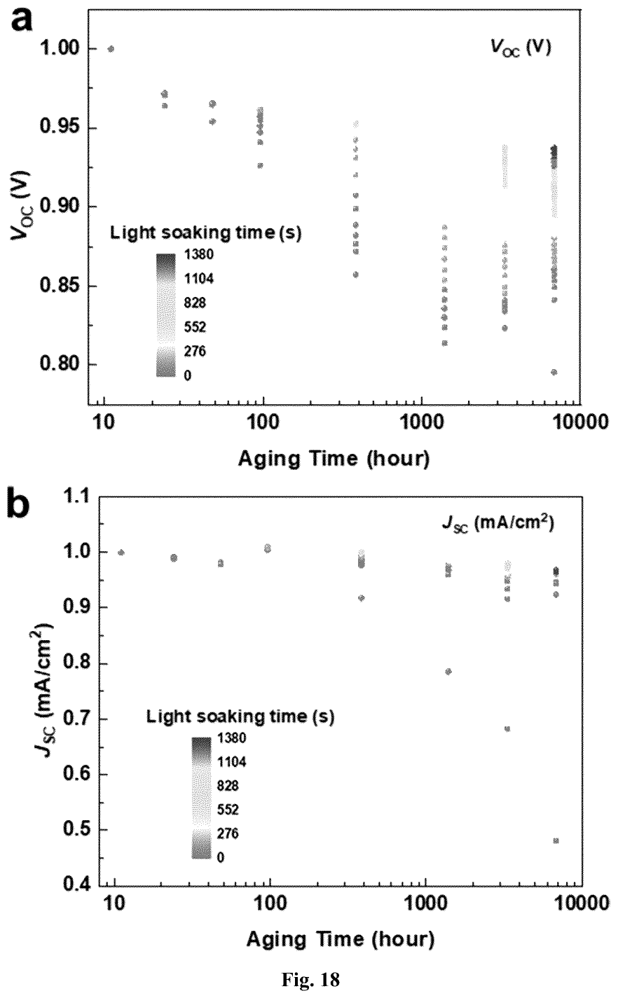

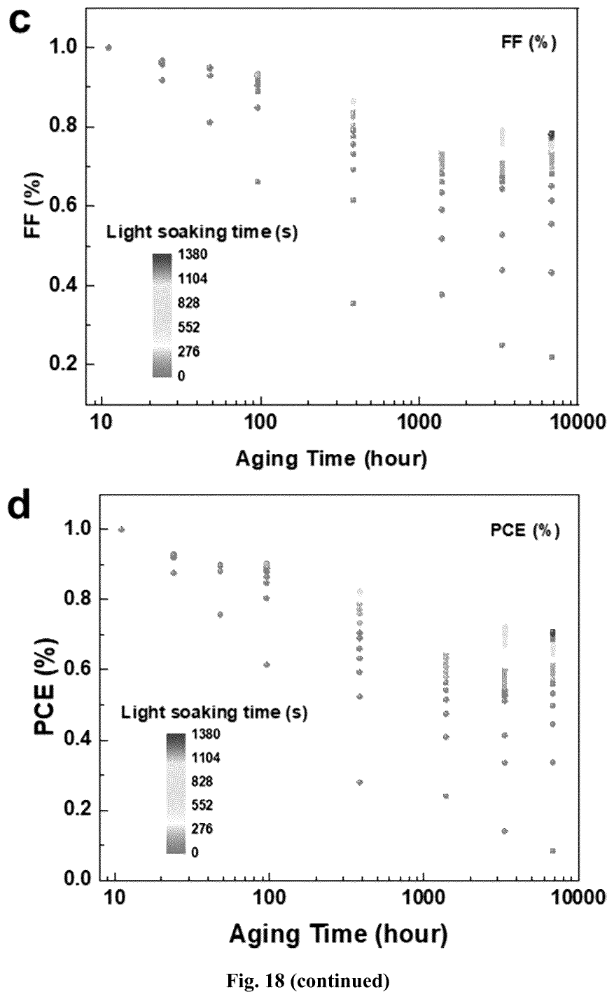

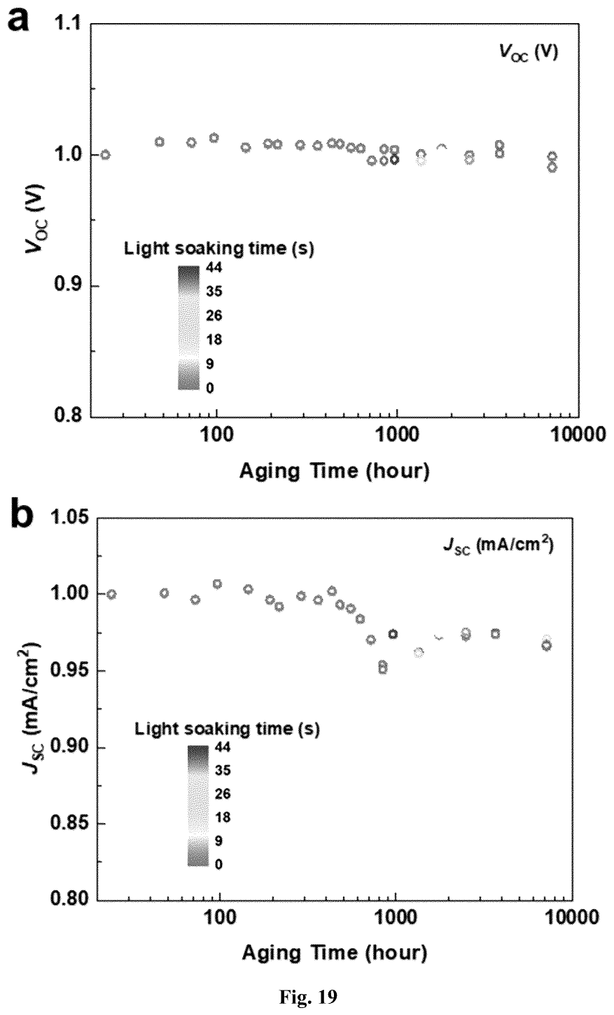

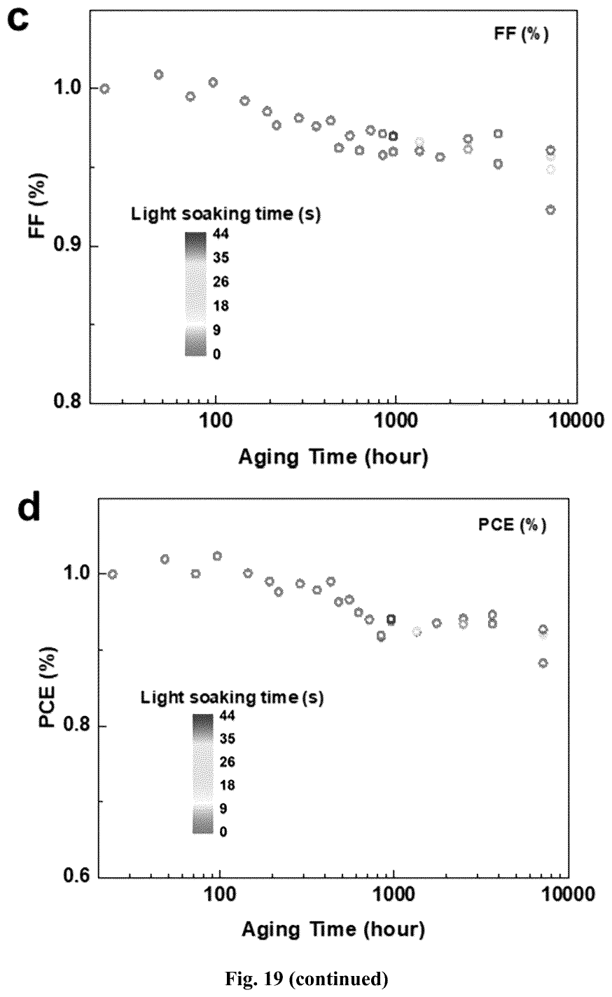

The stability of OPV devices based on ZnO and BHT@ZnO NPs under various aging conditions was assessed. Initially, the photovoltaic performance of unencapsulated devices was tested to determine the decay rate when aged in ambient condition (room temperature, 20-30% relative humidity). We observed interesting yet abnormal phenomenon of the light soaking effect on OPV J-V testing. The PCE variations are provided in FIG. 2d, while detailed parameters are summarized in FIG. 18. The freshly prepared devices using both ETLs exhibit no light soaking effect, but they manifest a growing light soaking effect after exposure to ambient conditions. And longer aging time exacerbates the light soaking effect. In ZnO-based devices, as the ambient condition aging time increases, not only the first scan tested PCE drop dramatically, the continues testing time need for reaching stable state also increase significantly. Specifically, after approximately 96 h of storage at ambient conditions, the ZnO-based device exhibits a 61.5% retention rate of its initial PCE at its first J-V scan (without soaking). Thereafter, the device's PCE gradually improves to 90.1% of the initial PCE after soaking under AM1.5G (100 mW cm⁻²) for about 292 s. Then, after 6840 h storing, the ZnO-based device shows a PCE lower than 10% of its initial value in the first J-V scan. The PCE is improved to 70.8% after soaking for 1380 s. The J-V curves variation of the ZnO-based device during the soaking time is provided in FIG. 2e. The device presents an initial S-shape during the initial few J-V scans. Three parameters including V_{OC}, J_{SC}, and FF are significantly increased by illumination. In contrast, the devices based on BHT@ZnO shows barely light soaking effects when even expose to ambient conditions for the long term (FIG. 19). After 7176 h storing, the device based on BHT@ZnO still shows 88.3% of initial PCE in its first J-V scan, then slightly increases to 92.8% after soaking for 34 s.

Metal oxide-based inverted OPV devices without encapsulation have been observed to exhibit light soaking effect that should be connected to the presence of oxygen species on the surface. In particular, O₂ and H₂O can be adsorbed, leading to electron extraction from the conducting band of the metal oxide and the formation of a depletion region and upward band bending. This upward band bending impedes electron extraction at the ZnO/bulk heterojunction (BHJ) or ZnO/ITO interfaces. During light soaking process, the photogenerated hole in ZnO recombines with the oxygen species, desorbing them and enabling improved performance. It also suggests that the significantly suppressed light soaking effect in BHT@ZnO-based devices can be attributed to the surface passivation of V₀⁺, which blocks the adsorption of oxygen species and prevents the formation of a depletion region and upward band bending.

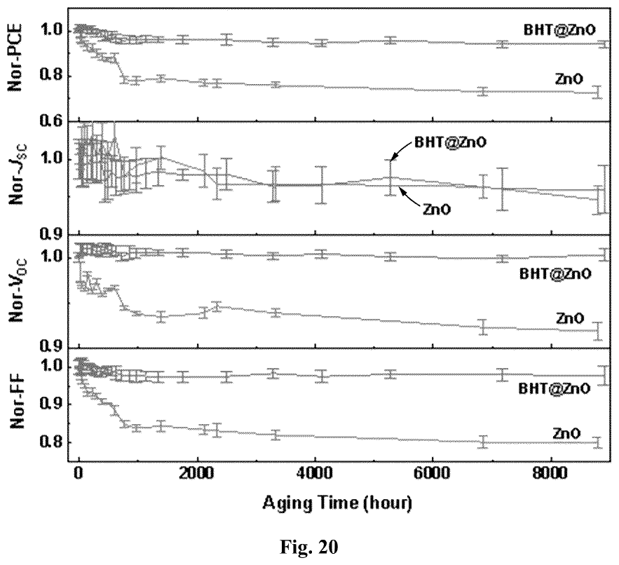

Although the PCE would be partially recovered after light soaking, the devices based on ZnO still show serious degradation after ambient aging. The detailed parameters of the PCE degradation after ambient storage and light soaking recovered are provided in FIG. 20. Specifically, a significant burn-in loss is observed which shows a PCE decay of 21% in the first 600 h. After storing at ambient dark conditions for over 8784 h, only around 72.9% of the initial PCE was retained in the devices based on ZnO NPs. In contrast, the devices based on BHT@ZnO retain about 92.8% of the initial PCE after over 8904 h. The rapid PCE decay is mainly associated with V_{OC} and FF. To figure out the charge recombination behavior of the fresh and aged devices, the V_{OC} variation versus light intensity was measured after light soaking (FIG. 2f). The aged device based on ZnO shows a higher ideality factor n of 1.42 than the fresh device (1.16) which suggest severer trap-assisted recombination occurs after aging at ambient condition. It indicates that the adsorption/desorption of oxygen species on ZnO surface is not fully reversible. The oxygen species would not fully desorb from the V_{O}⁺ after long-term ambient aging, even under long time illumination. In contrast, the n value of the BHT@ZnO-based fresh device exhibits a smaller n of 1.04 and slightly increases to 1.10 after aging which indicates reduced trap-assist recombination.

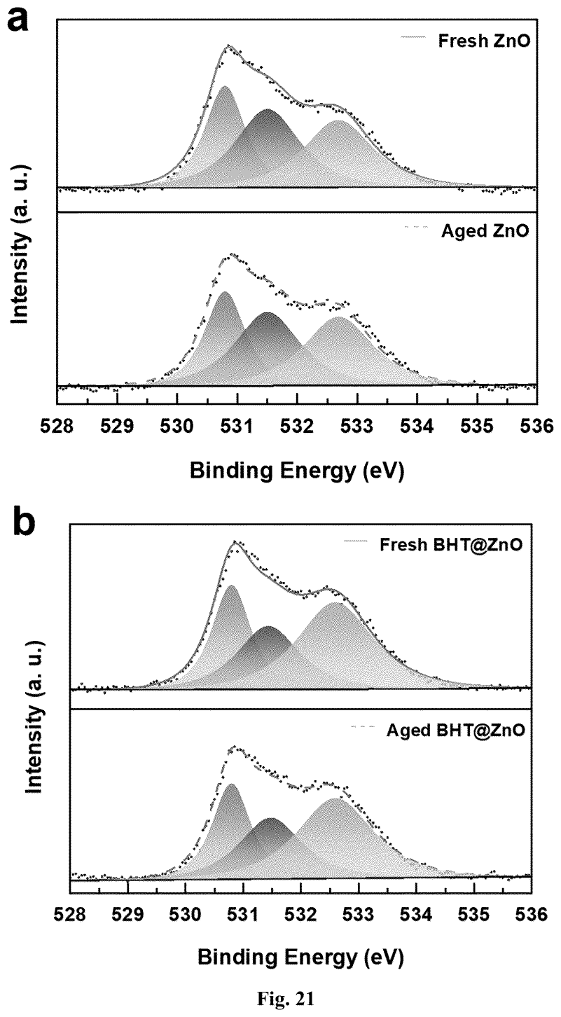

To prove the oxygen adsorption behavior, both the aged ZnO and the aged BHT@ZnO in ambient conditions were performed with XPS measurement (FIG. 21 and Table 1). Compared to the fresh ZnO film, the aged ZnO showed higher content of the surface adsorbed oxygen, which suggest the adsorption of the oxygen species from ambient conditions. While the surface adsorbed oxygen content of the fresh and aged BHT@ZnO are very similar, which demonstrates the modification of the BHT moiety can shield the Vₒ⁺, blocking the adsorption of oxygen species.

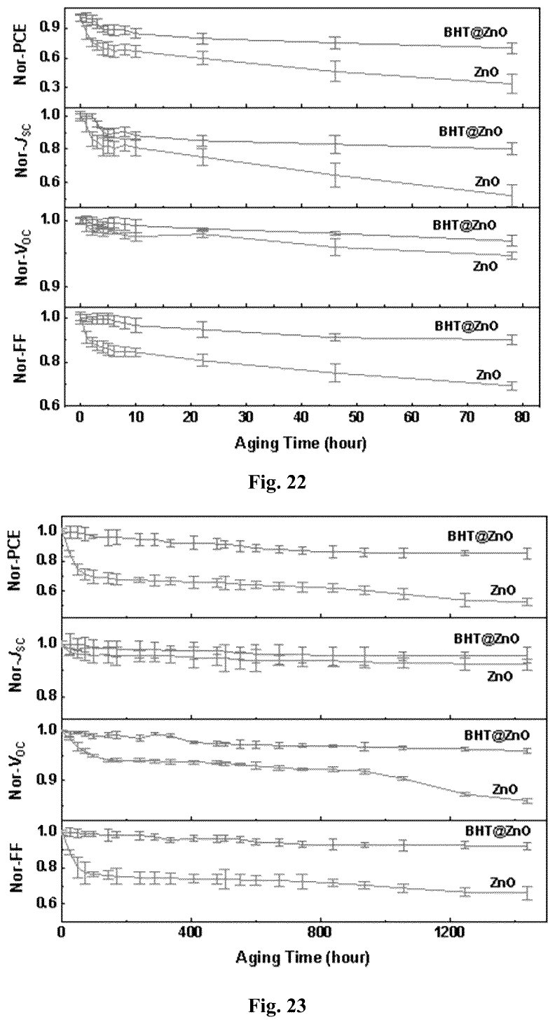

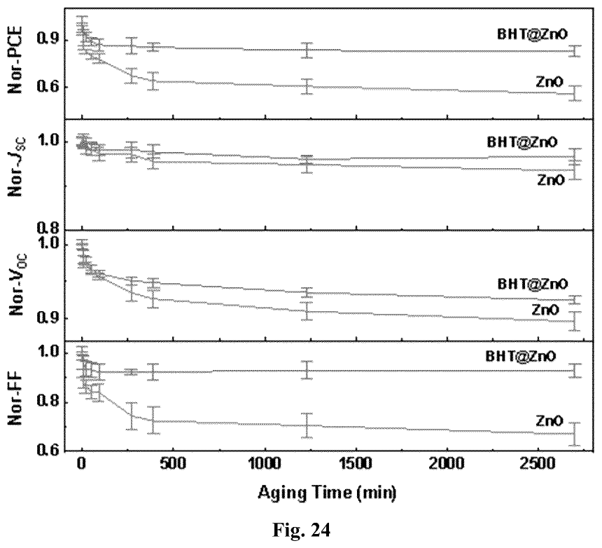

The devices were also aging under light exposure (100 mW cm⁻² white LED) with/without encapsulation at open circuit condition. After aging at ambient conditions for about 78 h, the ZnO-based device without encapsulation retains only 35% PCE of the pristine value, while the counterpart of the BHT @ZnO-based device is over 70% (FIG. 22). The encapsulated devices based on BHT@ZnO also show a significant improvement in the PCE retention increase to 85% after 1440 h 1-sun aging, vs. 52% in ZnO case (FIG. 23). Besides, the device lifetime was also tested at UV illumination. Under the UV exposure in N₂, the ZnO-based devices showed rapid degradation with only 56% PCE retention after 45 h, which is much lower than the counterpart of BHT@ZnO-based devices (83%) (FIG. 24).

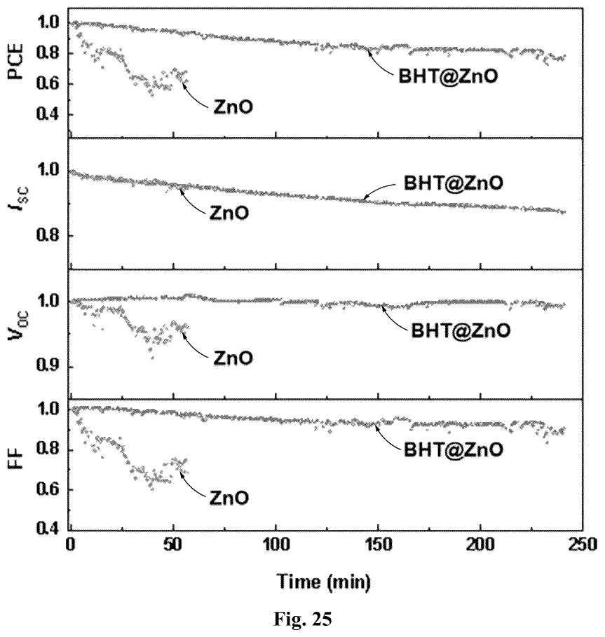

The operational stability of the devices was further tracked under MPP with continuously simulated 1-Sun illumination using LED arrays in ambient conditions. As shown in FIG. 2g and FIG. 25, the device based on ZnO ETL without encapsulation exhibited rapid PCE degradation, decreasing to about 60% of the initial value within 60 minutes. When ETL is BHT@ZnO, the OPV device showed stable PCE output, retaining over 75% of the initial value after 240 minutes of MMP aging without encapsulation. Meanwhile, encapsulated devices were tested in MPP conditions (FIG. 2h and FIG. 26). The device based on ZnO NPs had serious PCE decay, with the PCE decreasing to lower than 80% of the initial PCE after 580 hours. By contrast, the BTH@ZnO-based device retained 85.04% of the initial PCE after aging for 2207 hours. This demonstrates that the BTH capping could effectively protect the ZnO from ambient pressures and enhance the operational stability of the OPV devices.

### 4. Morphology Variation

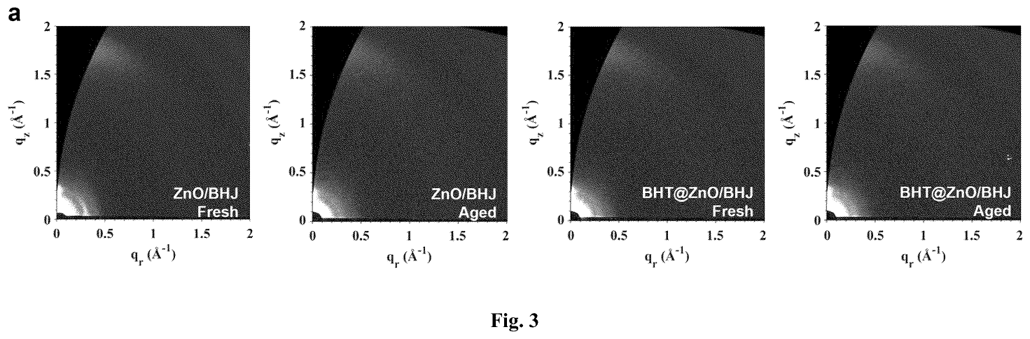

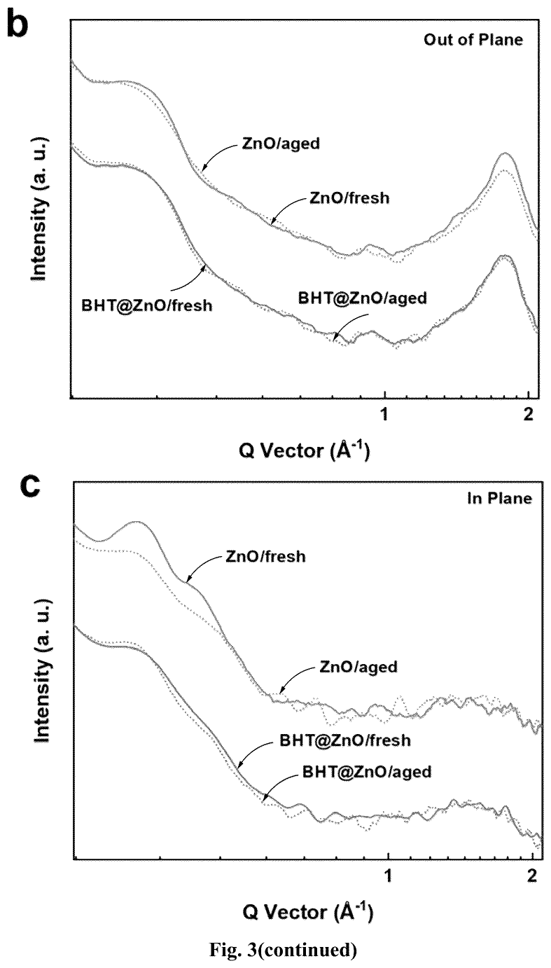

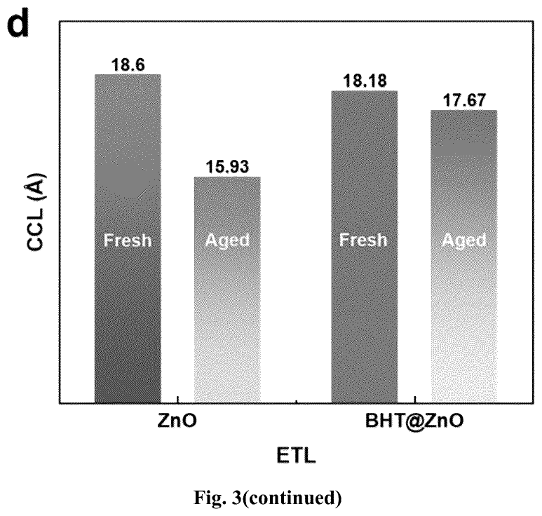

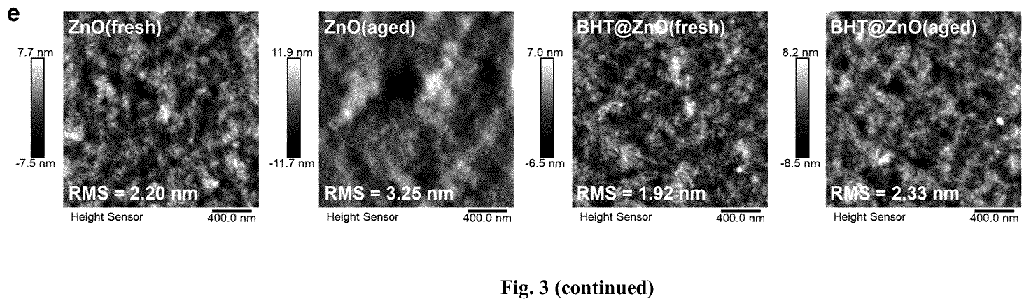

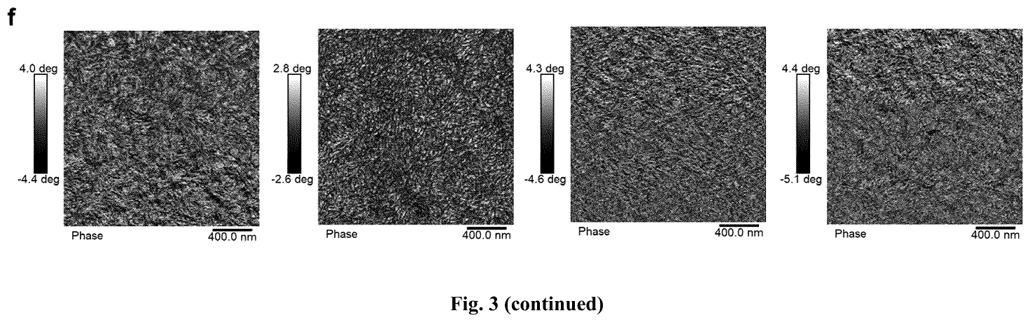

Grazing-incidence wide-angle x-ray scattering (GIWAXS) was used to detect the evolution of the BHJ layers based on ZnO and BHT@ZnO before and after aging under light illumination in ambient conditions. The images showed that both the ZnO/BHJ and BHT@ZnO/BHJ exhibited a face-on orientation with a π-π stacking (010) peak with a Q vector of 1.785 Å⁻¹ along the profile in the out-of-plane (OOP) direction and a lamellar stacking (100) peak with a Q vector of 0.295 Å⁻¹ along the profile in the in-plane (IP) direction (FIG. 3a). The fresh BHJ based on ZnO and BHT@ZnO ETL exhibited similar crystalline coherence length (CCL) of 18.60 Å and 18.18 Å, respectively (Table S4 in FIG. 38). After aging under UV illumination, obvious decay for both the π-π stacking peak and lamellar stacking peak were observed in the ZnO-based BHJ film (FIG. 3b-c). The CCL of the BHJ decreased significantly to 15.93 Å, indicating serious morphology variation/crystallinity degradation of BHJ after aging, which may originate from photoactive material decomposition. In contrast, BHT@ZnO-based BHJ exhibited mitigated decay in both the π-π stacking peak and lamellar stacking peak, with the CCL of 17.67 Å after aging, a slight drop from 18.18 Å in fresh (FIG. 3d). Furthermore, AFM revealed the morphology variation of the BHJ. Distinct nanofiber structures and a smooth surface with RMS roughness of 2.20 nm and 1.92 nm were observed in both BHJ coated on ZnO and BHT@ZnO, respectively (FIG. 3e-f). After aging under light illumination, the RMS of ZnO-based BHJ significantly increased to 3.25 nm, and the amplitude in phase images was obviously reduced, demonstrating the degenerated phase separation in BHJ. By contrast, both surface roughness and phase separation in BHT@ZnO-based BHJ remained almost unchanged after aging. The GIWAXS pattern and AFM image variation confirmed that BHT modification effectively suppress the degradation of the active layer under light illumination.

### 5. Radical-Induced Selective Catalytic Degradation of Polymer/NFA

The active layer degradation mechanism under light radiation is also investigated herein.

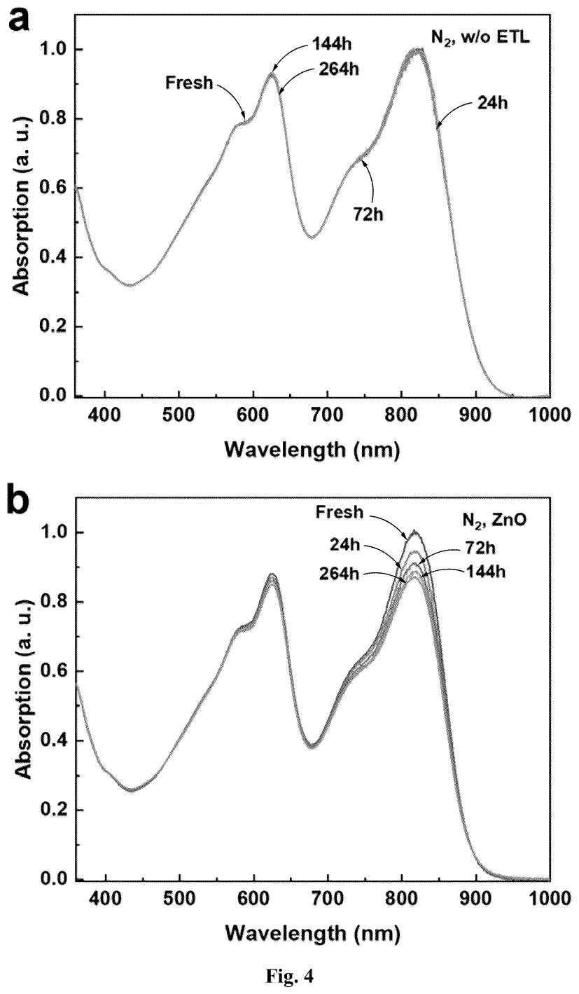

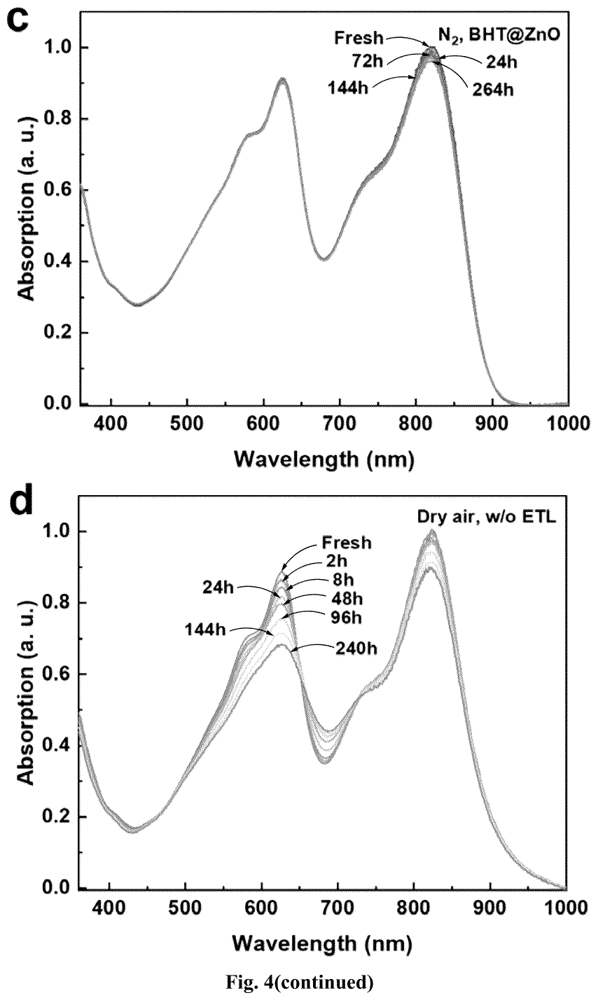

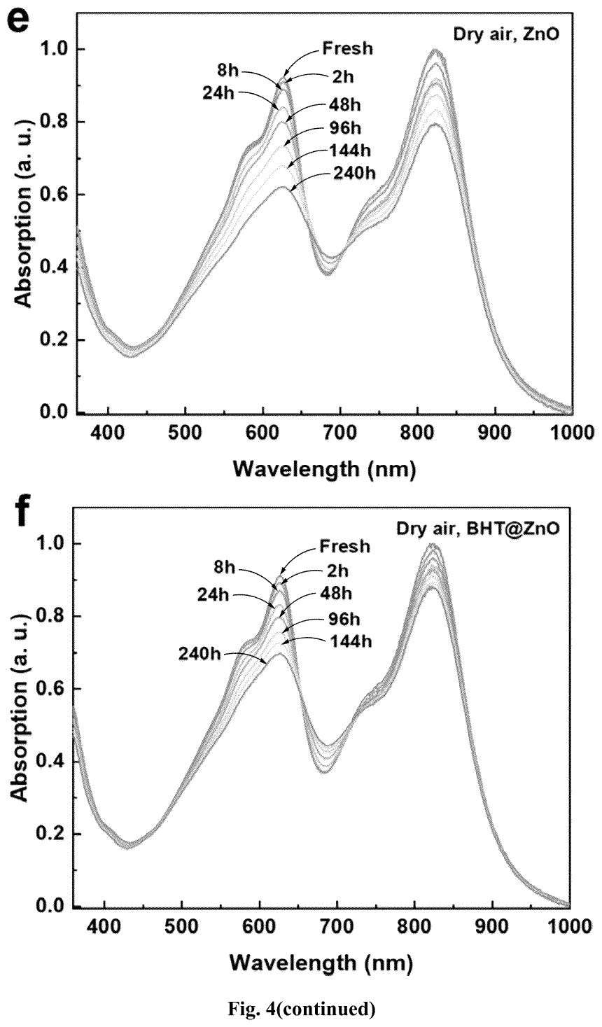

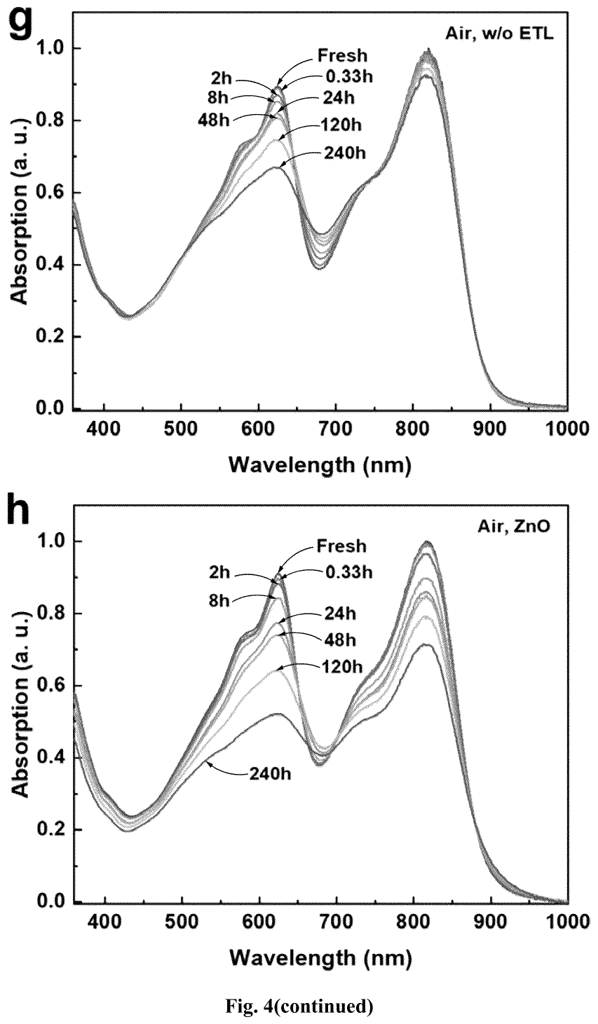

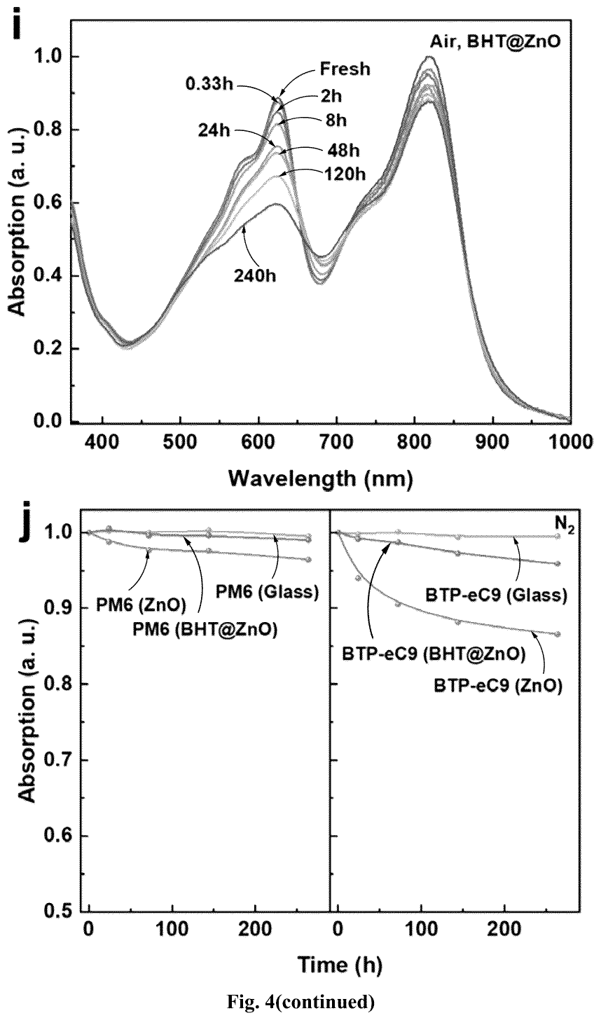

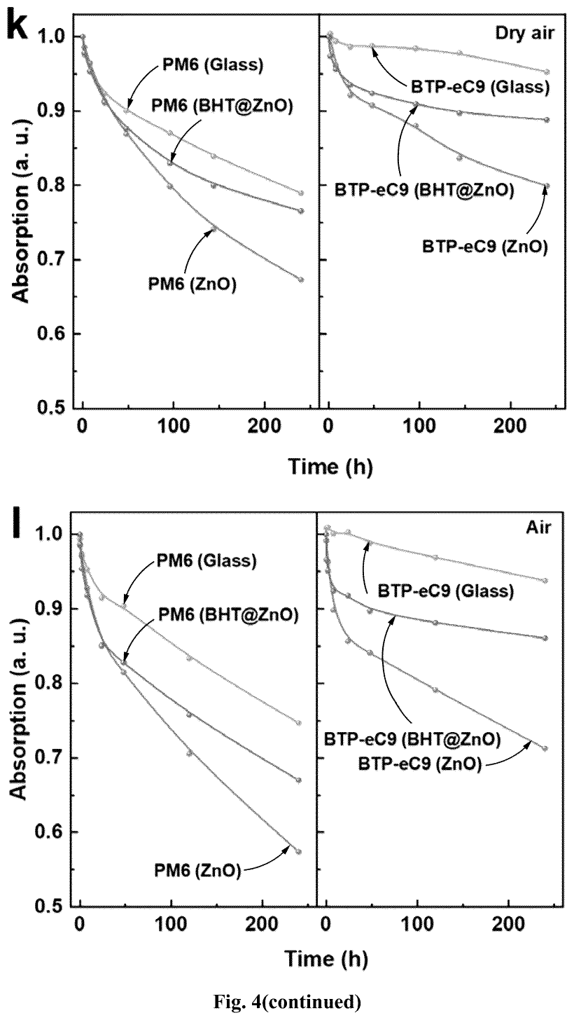

Trace amount of impurity in the active layer can cause defects to form, significantly affecting the performance of the photovoltaic system. Therefore, it is crucial to comprehend the molecular-level degradation of the active layer. UV-vis absorption spectra of the active layer after aging were measured. According to FIG. 4a, PM6: BTP-eC9 active layer coated on neat glass exhibited remarkable chemical stability, with unchanged absorption for both the donor (PM6) and acceptor (BTP-eC9) under continuous UV radiation in N₂ (O₂<10 ppm, H₂O<10 ppm). When coated on ZnO, the absorption of PM6 remained almost unchanged, while the absorption of BTP-eC9 was noticeably weakened, remained ca. 86% of initial intensity after UV radiation in N₂ as displayed in FIG. 4b. It can be inferred that when coated on ZnO ETL, the decay of BTP-eC9 is due to oxidation by hydroxyl radicals (HO·) generated from the adsorbed hydroxyl ion (OH⁻) on the ZnO surface. The aging profile of the blending film suggests that the polymer donor PM6 is not oxidized by HO·, implying that HO· selectively oxidizes BTP-eC9.

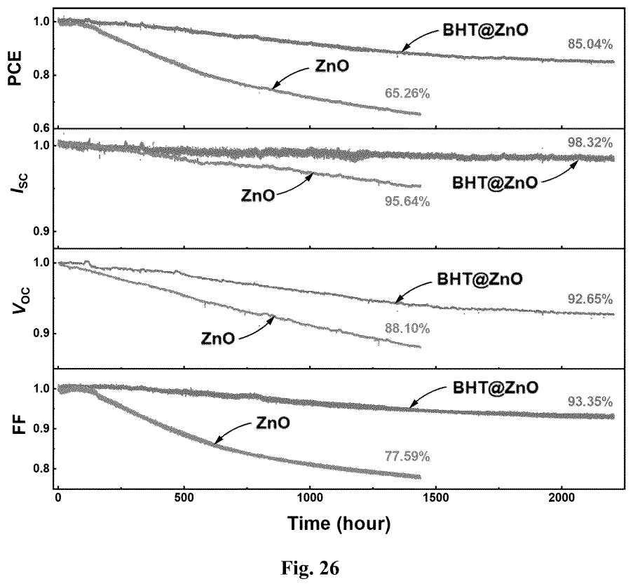

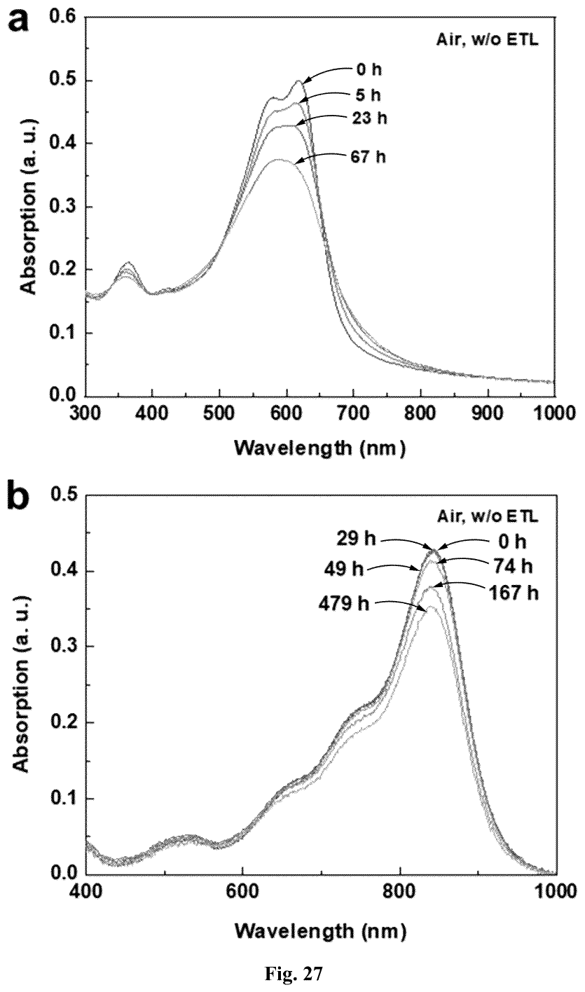

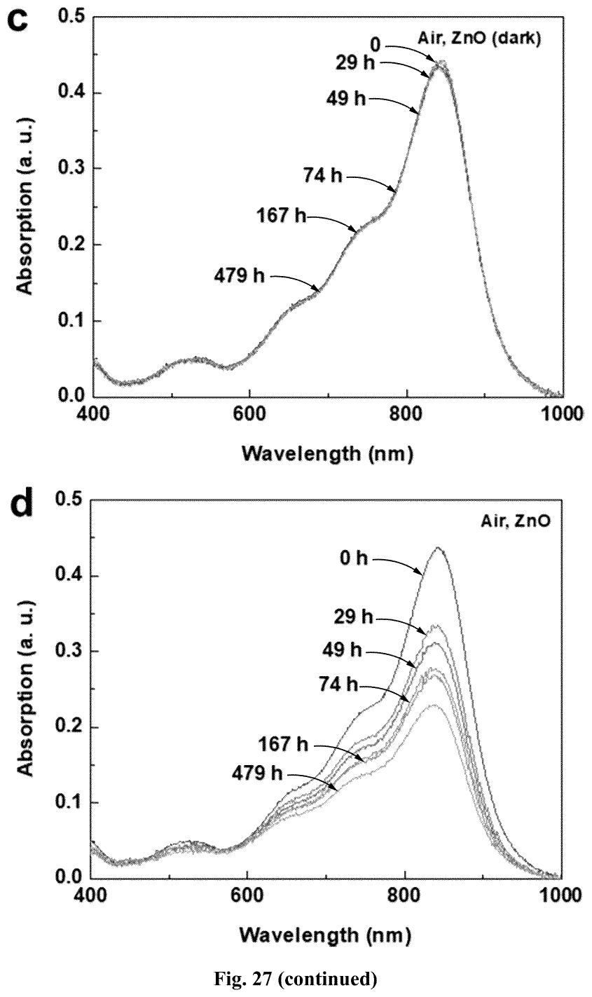

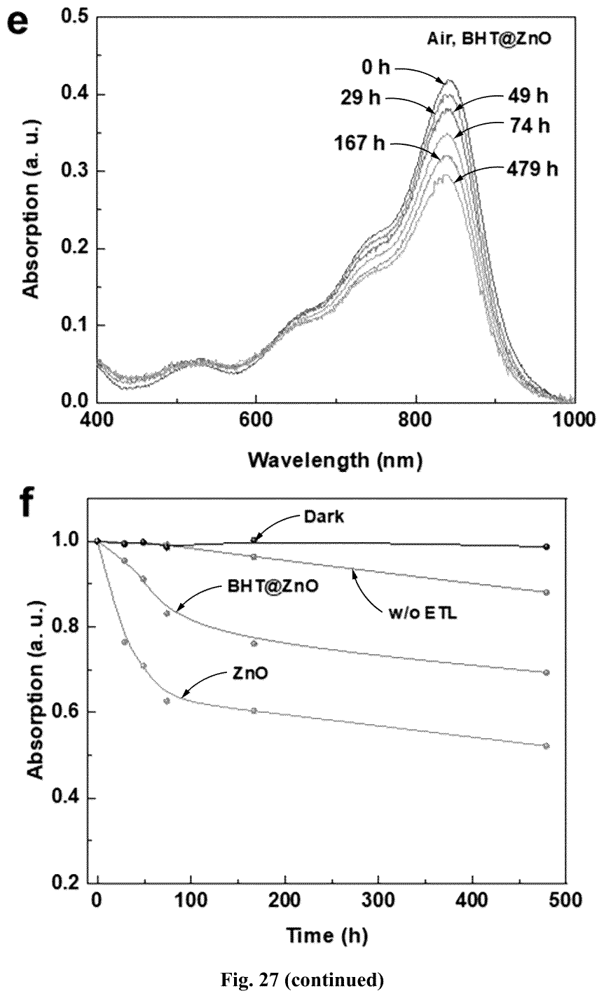

When aging on glass at dry air (O₂ content≈19%, relative humidity≈1%), the absorption decay of active layer was severer than under UV/N₂ condition. However, opposite to UV/N₂, the degradation of PM6 was stronger than BTP-eC9 under UV/dry air. According to FIG. 4d, PM6 suffered from obvious absorption decrease (80% of initial intensity after 240 h), even if without any ETL. We speculated the strong degradation of PM6 under UV/ambient was due to being oxidized by superoxide anion radical (—O₂), which could be generated from O₂/UV condition. Moreover, moderate decay of BTP-eC9 was observed (95% of initial intensity after 240h). Same samples were also aged at humid air (relative humidity≈60-70%) and showed similar absorption decay (75% and 94% retention for PM6 and BTP-eC9 after 240h aging) (FIG. 4g). It indicated that H₂O in air may not participate in active layer decay process under UV without ETL. So H₂O— participated transformation from ·O₂⁻ to HO· would not occur in this stage. In addition, the pure BTP-eC9 showed higher UV resistance to pure PM6 (FIG. 27). In this case, we concluded that the oxidation of ·O₂⁻ to PM6 is much higher than BTP-eC9. In other words, the ·O₂⁻ exhibited selective oxidization to PM6 rather than BTP-eC9.

When aging on ZnO in air, both the PM6 and BTP-eC9 suffered severer absorption decay than their counterparts without ETL (FIGS. 4e and 4h). As we mentioned ·O₂⁻ and HO· were the culprit for PM6 and BTP-eC9 degradation, respectively, severer absorption decay means more ·O₂⁻ and HO· generated due to the presence of ZnO/UV radiation. Specifically, the electron in valence band of ZnO was excited to conducting band. The O₂ and H₂O adsorbed on the surface Vₒ⁺ of ZnO could extract the electron in conducting band and hole in valence band, then generate extra ·O₂⁻ and HO·, respectively. Furthermore, the BTP-eC9 experienced stronger absorption decay in humid air compared to dry air, suggesting that H₂O-participated HO· generation could occur with the existence of ZnO ETL (FIG. 4h).

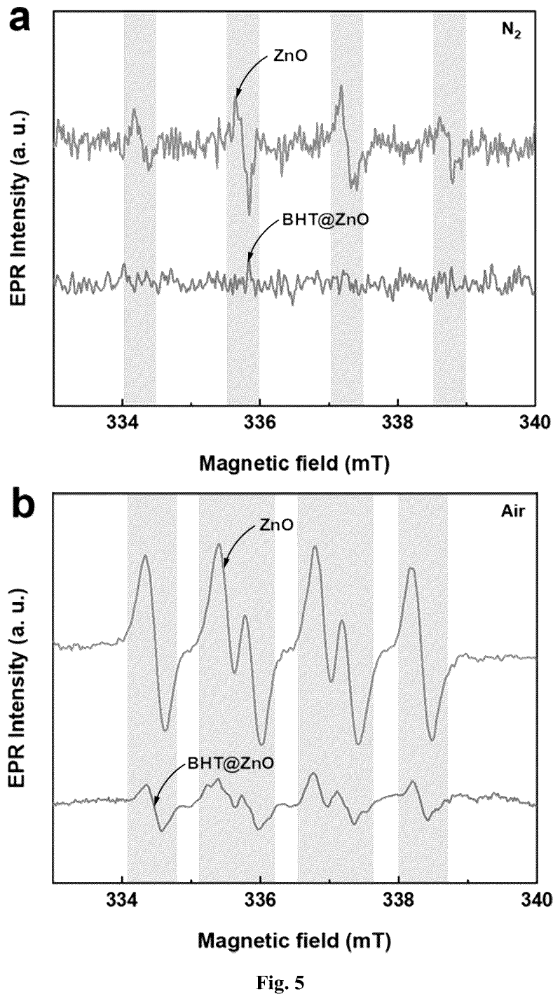

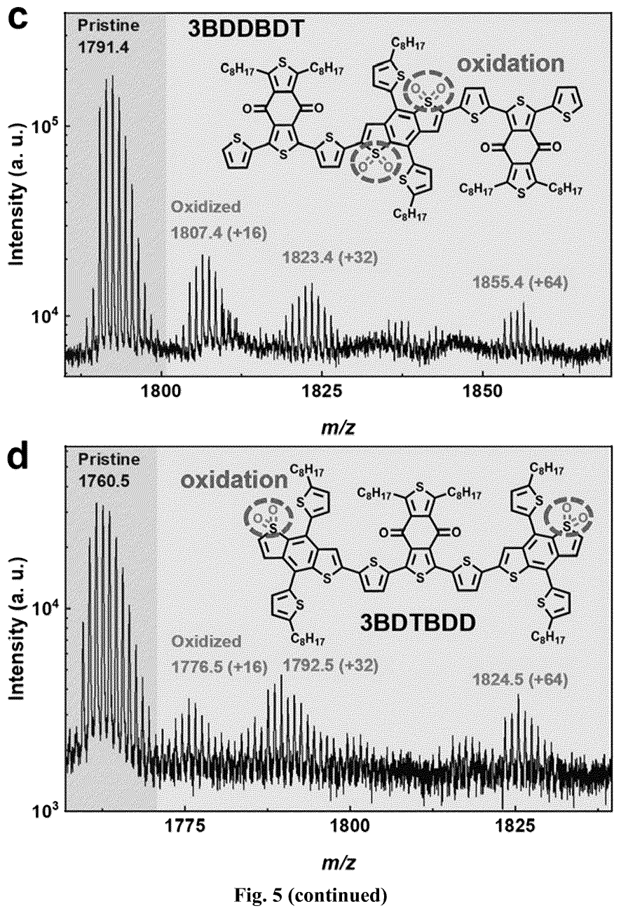

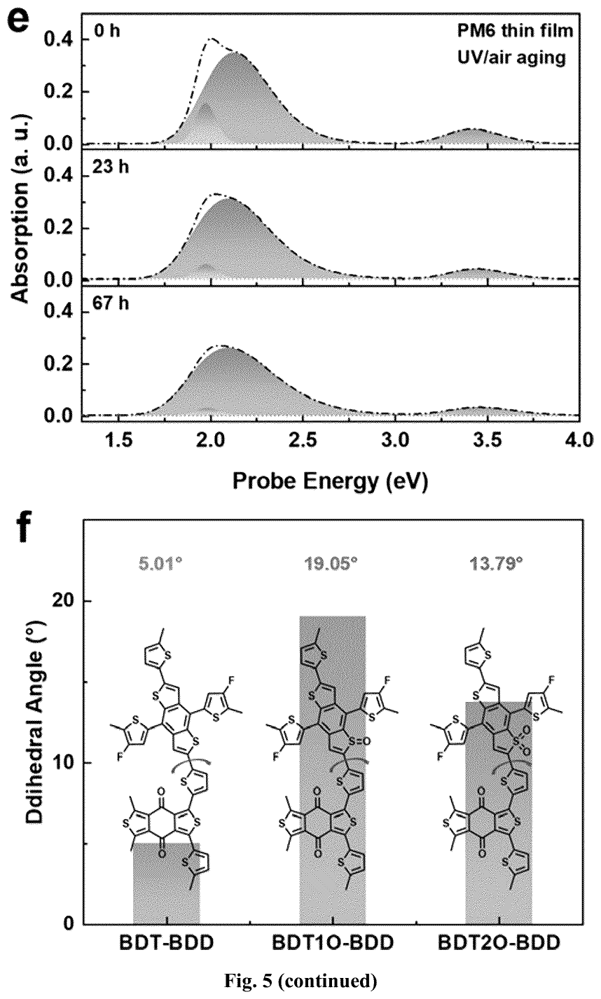

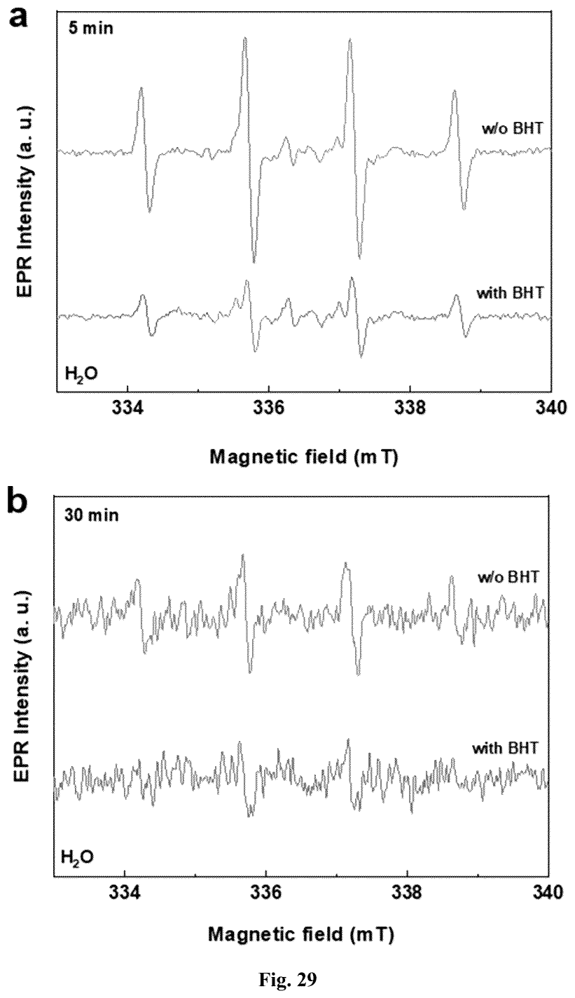

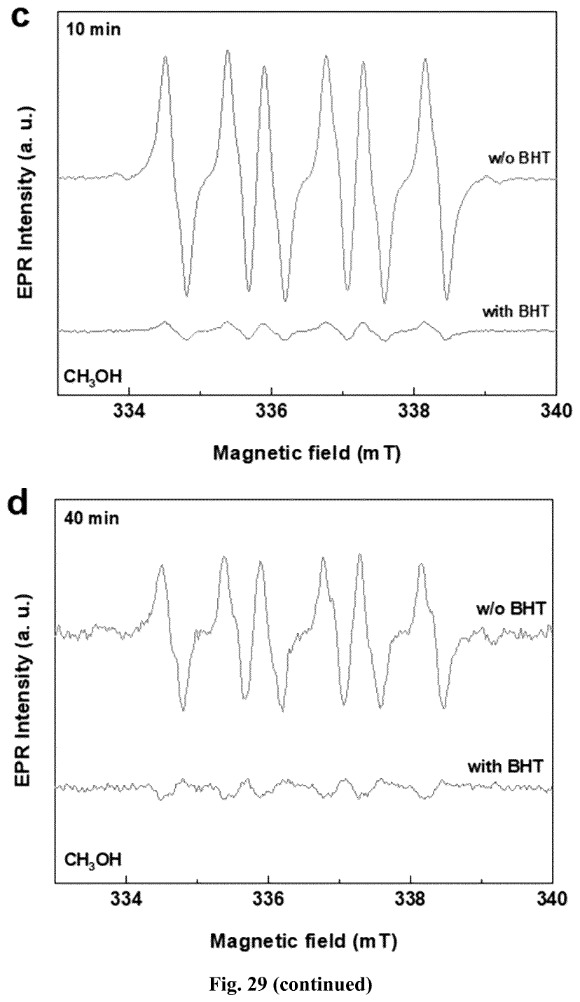

When replace ZnO with BHT@ZnO, both the PM6 and BTP-eC9 exhibited higher absorption retentions at the same aging states (FIG. 4j-4l), which indicates the ·O₂⁻ and HO· were suppressed with the existence of BHT@ZnO. Based on this, EPR was employed to detect the generation of HO· and ·O₂⁻ under UV/N₂ and UV/ambient, respectively, to prove our speculation. 5,5-Dimethyl-1-pyrroline-N-oxide (DMPO) was used as the radical trap. As observed in FIG. 28, ZnO did not generate any radicals under dark conditions in both N₂ and ambient. However, ZnO showed notable HO· signals under UV/N₂, and high ·O₂⁻ signals were observed under UV/ambient (FIG. 5a-5b). The ·O₂⁻ signals were too strong, covering up the HO· signals under UV/ambient. In BHT@ZnO samples, both the HO· and ·O₂⁻ signals were suppressed. This could be attributed to two reasons as follows. First, the passivated V_{O}⁺ on BHT@ZnO surface decreased the adsorption of residual hydroxyl ion, O₂ and H₂O (in ambient), thus decreasing the source of HO· and ·O₂⁻. Secondly, the radical scavenging ability of BHT could effectively capture the radicals, cutting off the degradation process of photoactive materials of OPV. Besides, to further demonstrate the radical scavenging capability of the BHT, BHT-acid was added into the Fenton's reagent with a concentration of 0.5 mg/ml. In Fenton's reagent, HO· was generated in H₂O solution, while ·O₂⁻ was generated in methanol solution. The Fenton's reagent was prepared as the aforementioned procedure. According to FIG. 29, the intensities of EPR signals for both HO· and ·O₂⁻ based on samples with BHT are obviously lower than the sample without BHT during the whole reaction. Apparently, the reduction of the ROS is scavenged by the BHT.

Previous research has suggested the degradation products of the NFA, but the other key component—polymer donor is yet to be determined. To determine the degradation path of the polymer donor PM6 in the presence of ·O₂⁻, it is necessary to address the challenge of characterizing the molecular variation of the polymer itself, which is hampered by its undefined structure, including molecular weight and number of repeating units. To overcome this limitation, we employed two oligomers, namely 3BDDBDT and 3BDTBDD, both of which have well-defined structures, to simulate the degradation process of PM6 (or PBDB-T). These oligomers serve as simplified versions of PM6 and are found to exhibit poor photo stability (FIG. 30). To analyze the oxidation products of the two oligomers after aging under UV/air, we employed matrix-assisted laser desorption/ionization time-of-flight/time-of-flight mass (MALDI-TOF/TOF-MS) spectroscopy (FIG. 5c-d). In the spectra, we observed distinct peaks corresponding to the initial molecular weights (m/z) of 1791.4 for 3BDDBDT and 1760.5 for 3BDTBDD (blue area). Furthermore, we observed a series of peaks with m/z values of 1807.4 (1791.4+16), 1823.4 (1791.4+32), and 1855.4 (1791.4+64) in 3BDDBDT film. Similarly, m/z values of 1776.5 (1760.5+16), 1792.5 (1760.5+32), and 1824.5 (1760.5+64) were observed in 3BDTBDD film. These peaks indicate that the aged products of both oligomers exhibited an increased molecular weight that corresponded to multiples of 16. Based on these findings, we speculated that the aged products of the oligomers contained sulfoxide or sulfone. These compounds are the products of thiophene oxidized by ·O₂⁻ or ·O₂H.

In the case of the BDT unit, which is conjugated with an aromatic group such as benzene, the sulfur atoms are more prone to oxidation compared to the sulfur atom in thiophene. Therefore, we concluded that the oxidized unit in both aged oligomers (3BDDBDT and 3BDTBDD) as well as the polymer (PM6) was the BDT unit, which was oxidized to form the BDT (n)oxide (BDTnO) (n=1-4) (FIG. 5c-d inset). This finding provides an explanation for the significant photo-bleached behavior observed in polymer donors that contain the BDT unit, such as D18 and PTB7, as well as the good photo-stability observed in non-BDT-contained polymer donors like PTQ10.

Although the BDTnO-contained matters may not be the final decomposed product, it still plays a decisive role in material properties. On the one hand, the oxidation process from thiophene in BDT to the sulfoxide or sulfone would completely convert the electron-donating property to an electron-accepting property. This would lead to the destruction of electron donor/acceptor alternative units in PM6 backbone and the loss of the intramolecular charge transfer process⁵⁵⁻⁵⁹⁻⁶⁰. On the other hand, the sp² hybridization in thiophene changes to sp³ hybridization in oxidized thiophene, weakening the π-π stacking of donor materials by increased intermolecular steric hinder. The reduced π-π stacking was already observed in the GIWAXS measurement. Consequently, these preliminarily aged BDTO-contained products significantly impact the properties of initial donor materials, which greatly deteriorates the photovoltaic performance.

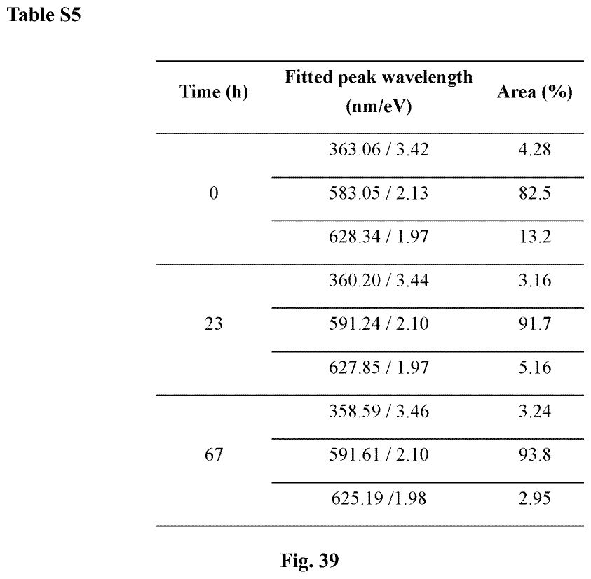

For further correlating the molecular packing variation by PM6 oxidation, the absorption spectra of pure PM6 thin films with different aging states were investigated (FIG. 5e). The peak of 1.97 eV indicated the order packing phase while 2.11 eV indicated the amorphous phase. The order phase exhibited an area ratio of 13.2%, 5.16%, and 2.95% for fresh, 23 h, and 67 h aged PM6, respectively (Table S5 in FIG. 39). The decreased order phase and increased amorphous phase of PM6 should be attributed to the deteriorated conjugated plane in the polymer main chain.

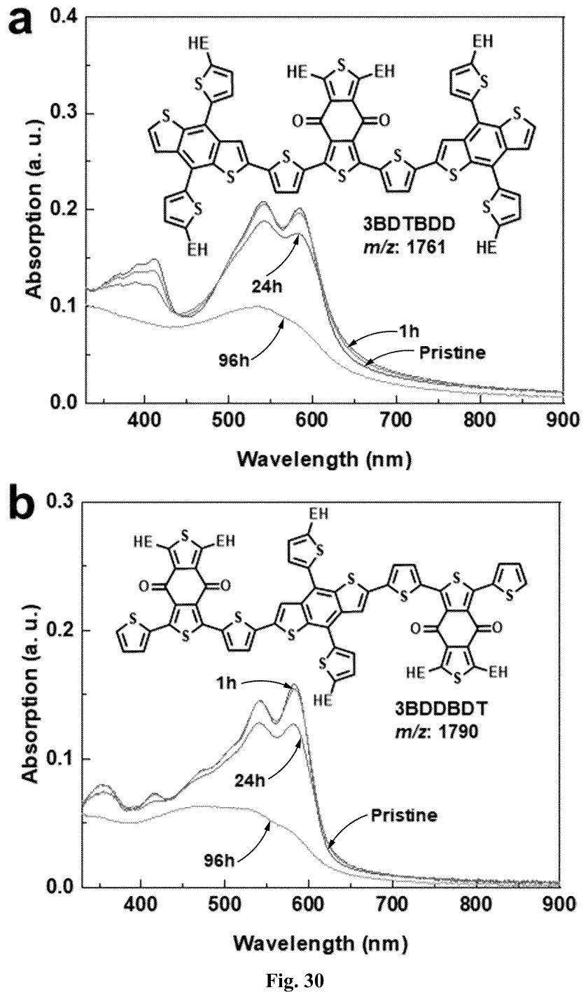

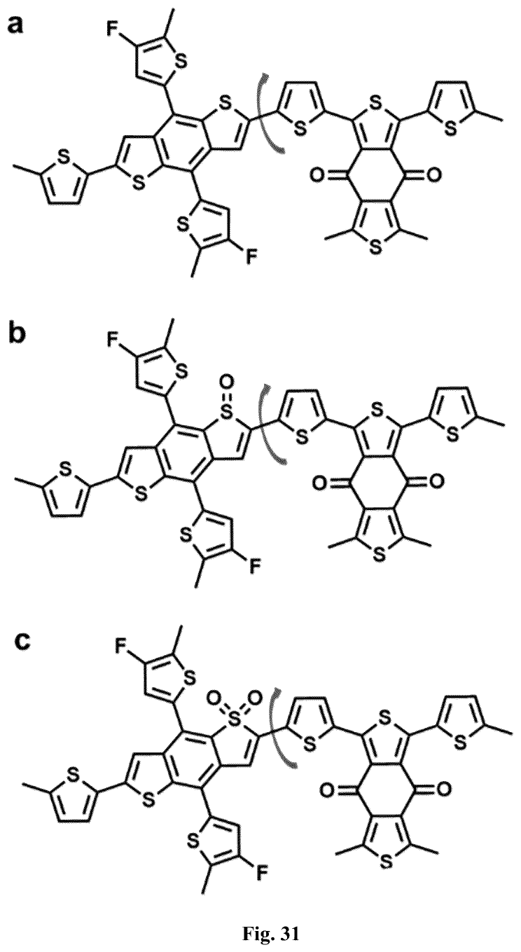

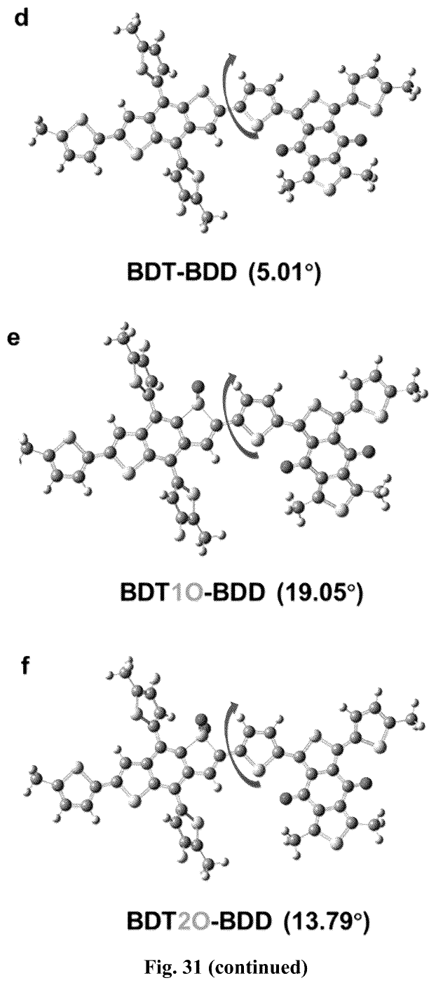

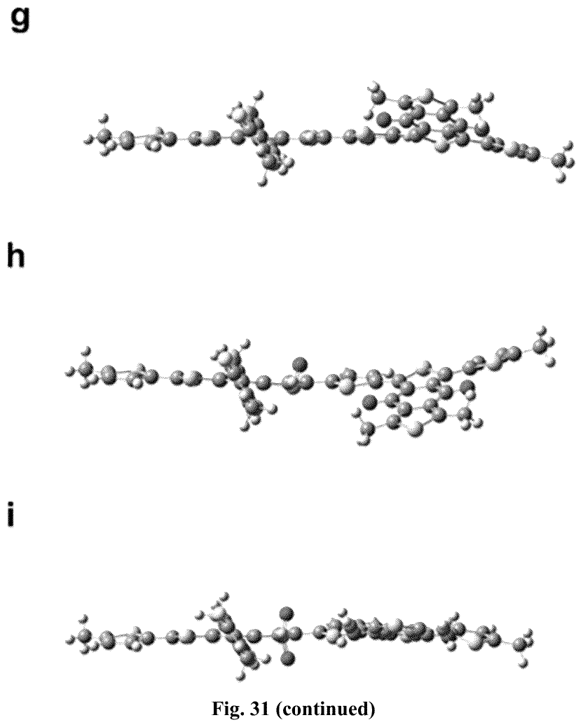

Notably, recent research revealed that the stronger backbone twist occurred in bleached PM6 film after UV/ambient conditions, but the origin of this backbone twist is still unknow. Here we calculated the dihedral angle between BDT and BDD in BDT-BDD, BDT1O-BDD, and BDT20-BDD molecules, respectively, by DFT simulations to study the change of geometry structures in PM6 backbone (FIG. 31). The dihedral angle between BDT/BDD moieties is 5.01°, while the oxide products, BDT1O-BDD and BDT20-BDD exhibited larger dihedral angle of 19.05° and 13.79° (FIG. 5f). It indicated the enlarged dihedral angle inside the backbone of PM6 was due to the generate of BDTnO (n=1-4). This enlarged dihedral angle (twisted backbone) also deteriorate the conjugated plane and convert the order phase in PM6 to amorphous phase. Our work is consistent with previous simulated result and provided more and deeper evidence for explaining this UV/ambient induced backbone twisting of PM6¹⁸. For future donor materials designing, it is necessary to exploring other electron donor monomer for replacing BDT unit aiming to improve the stability in molecule level.

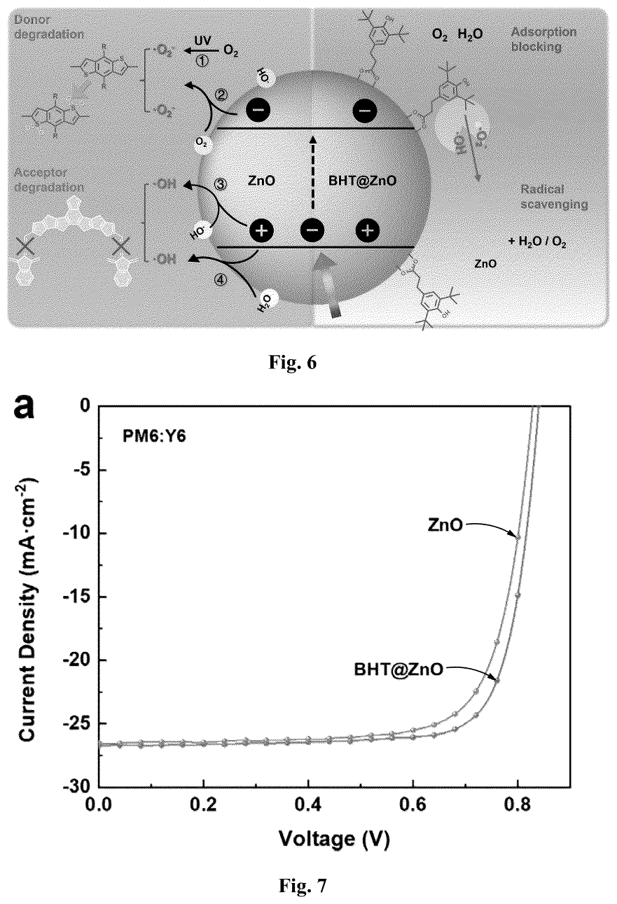

FIG. 6 presents a summarized schematic diagram illustrating the radical generation and scavenging processes involved in the stability issue of the active layer under light illumination and the function of BHT@ZnO. First, in the absence of ZnO, UV illumination of O₂ leads to the generation of ·O₂⁻ (Process 1). Second, when ZnO is excited by UV, O₂ adsorbed in Vₒ⁺ extract the electrons in the conducting band and generate additional ·O₂⁻ (Process 2). PM6, the donor material, is easily oxidized by ·O₂. Additionally, UV excitation of ZnO allows H₂O adsorbed in Vₒ⁺ extract the hole in the valence band and generate HO· (Process 3). Lastly, the adsorbed hydroxyl ion in Vₒ⁺ extract the hole in the valence band of ZnO and generate extra HO· (Process 4). The strongly oxidizing HO· selectively decomposes non-fullerene acceptors. The adsorption of O₂ and H₂O from the external environment by Vₒ⁺ contributes to the light soaking effect and aggravate the materials deterioration. For the BHT@ZnO ETL, the carboxyl groups effectively passivate V₀⁺, and two hydrophobic tert-butyls in BHT prevent the adsorption of O₂ and H₂O, thereby blocking Processes 2 and 3. The residual hydroxyl ions are also neutralized by BHT-acid, blocking Process 4. Moreover, BHT efficiently scavenges the inevitably generated HO· and ·O₂⁻, thus preventing the decomposition of both donors and acceptors.

In summary, it is discovered that under light illumination in N₂ or air, the degradation trends of donor (e.g., PM6) and acceptor (e.g., BTP-eC9) are opposite. Specifically, the acceptor shows higher degradation rate in N₂, while the donor exhibits higher degradation rate in air on the metal oxide layer. Subsequently, the selectively catalytic-degradation pathways of acceptor and acceptor by superoxide radical (·O₂—) and hydroxyl radicals (·OH) respectively are established. It is identified that the ·O₂⁻-induced benzo[1,2-b:4,5-b0]dithiophene (BDT) oxidation is the main factor in PM6 degradation failure. Consequently, BHT@ZnO effectively suppressed the ROS, including ·O₂⁻ and ·OH, by passivating the surface defects (reducing ROS generation) and scavenging the generated ROS. This provided excellent protection for the active layer materials. Hence, the present disclosure not only achieved high efficiency and stable OPVs, expanded understanding of the active layer material degradation mechanism, but also provided guidance for future OPV materials and device design towards commercially viable OPV technology.

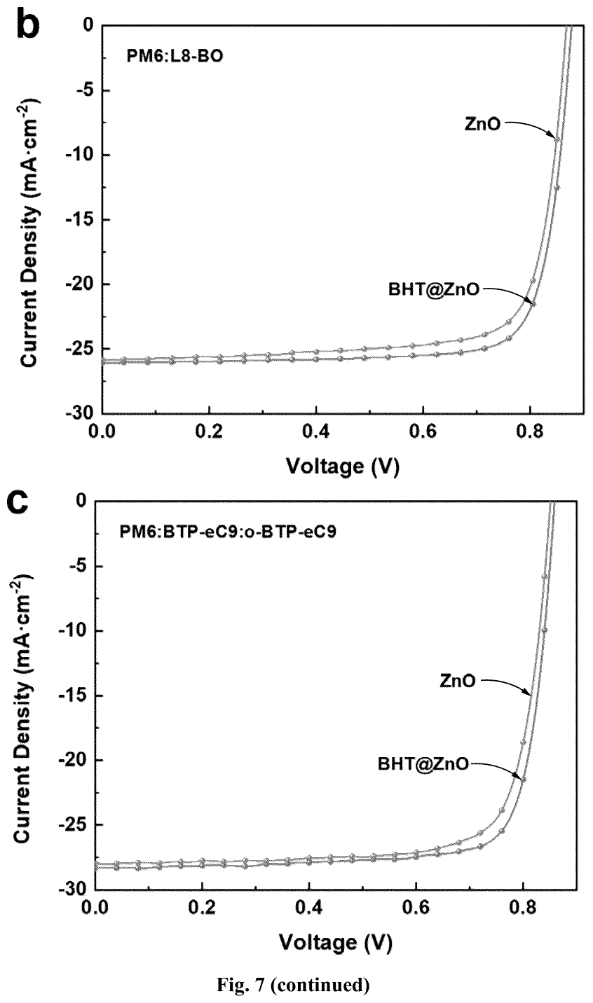

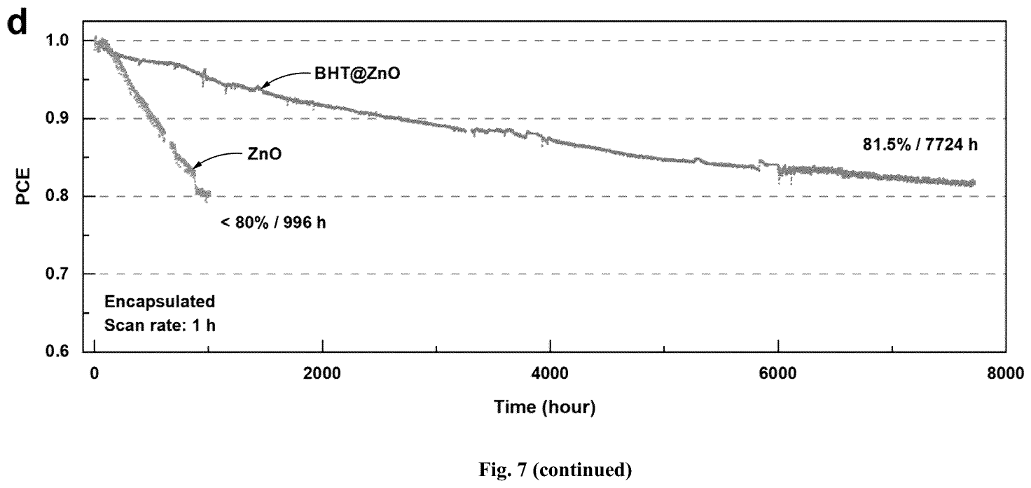

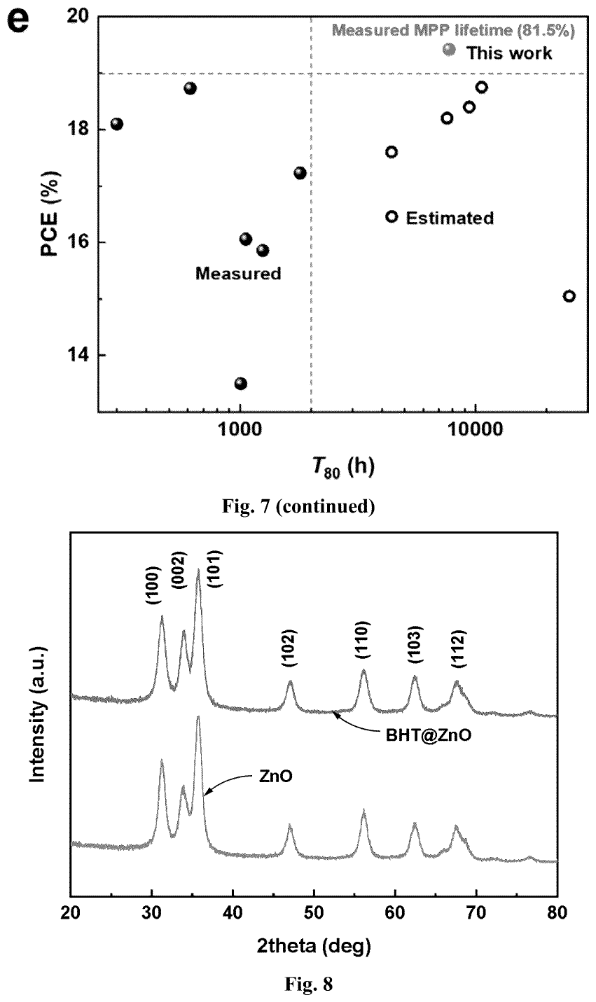

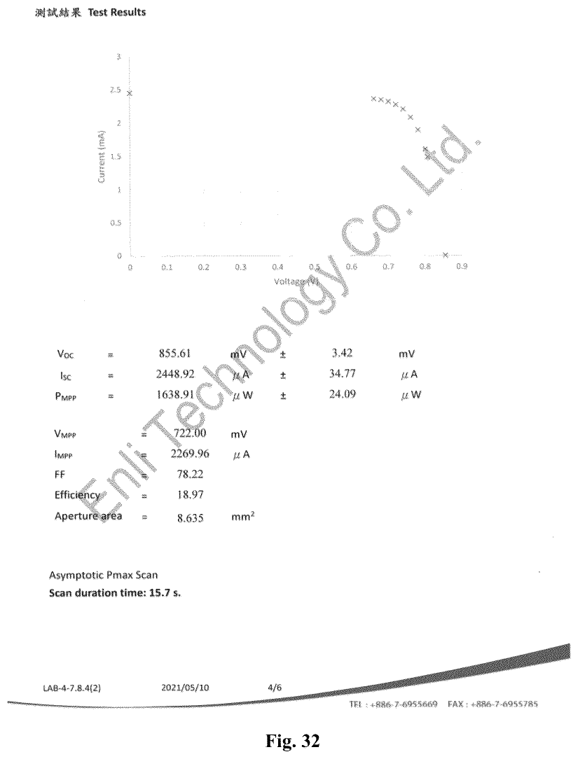

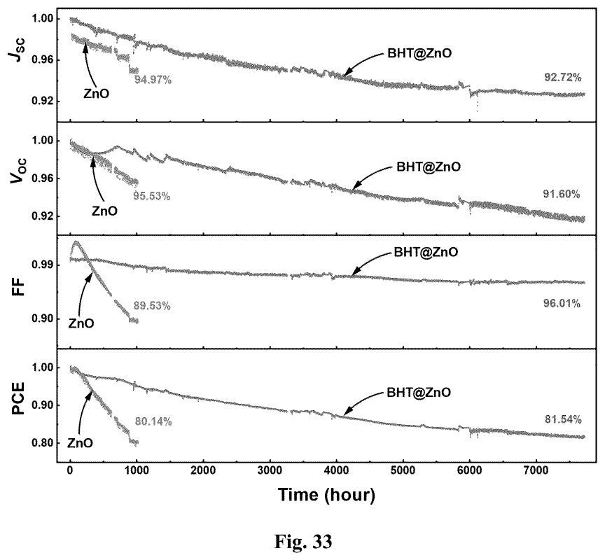

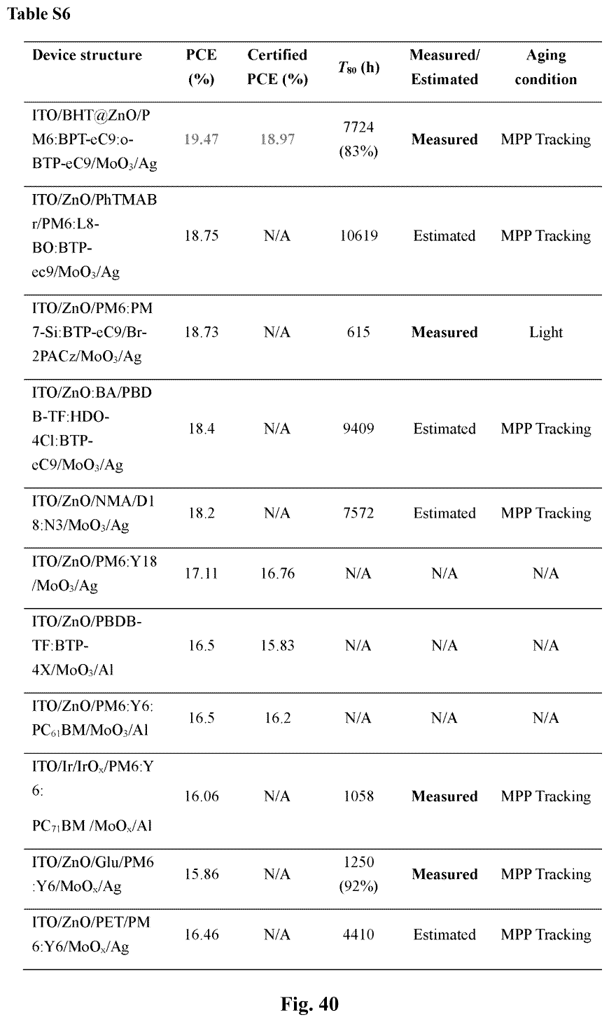

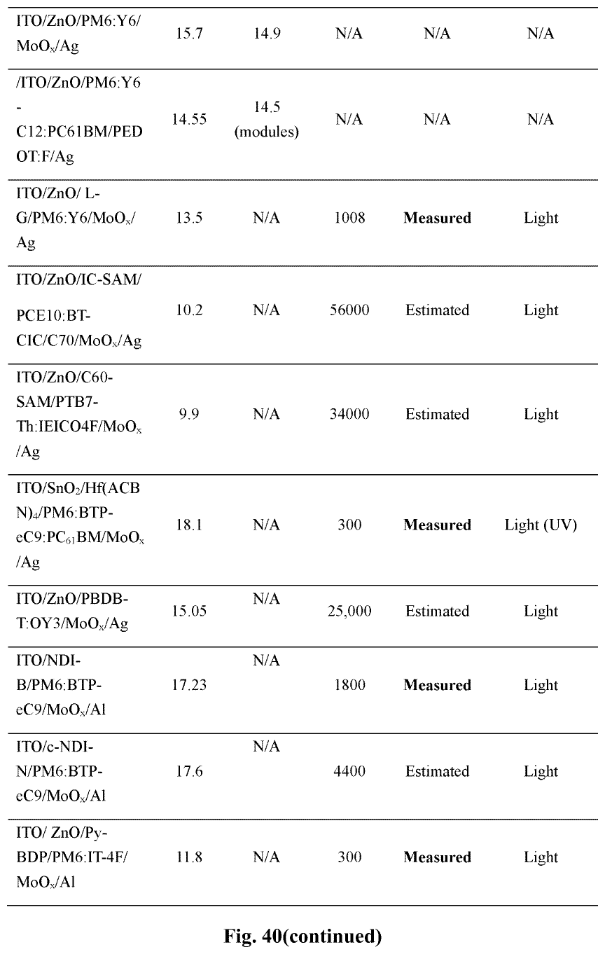

Finally, for proving the versatility of BHT@ZnO, three systems including PM6:Y6, PM6:L8-BO, and PM6:BTP-eC9:o-BTP-eC9 have been employed as active layers in inverted OPV. As seen in FIG. 7a-7c and Table 1, the devices based on BHT@ZnO performed better PCE of 17.52% and 18.45% of landmark systems PM6:Y6 and PM6:L8-BO, respectively, which were obviously higher than the ZnO-based devices (16.49% and 17.38%). Moreover, we applied a ternary system PM6:BTP-eC9:o-BTP-eC9 which is reported by our group with a high efficiency approaching to 20% (regular structure) into inverted structure⁶³. The corresponding inverted BHT@ZnO-based device exhibited a record-high efficiency of 19.47% with a V_{OC} of 0.859 V, J_{SC} of 28.39 mA·cm⁻² and FF of 79.87%. The device was certificated by Enli Tech Optoelectronic Calibration Lab (ISO/IEC 17025:2017 accredited Calibration Lab) with an efficiency of 18.97% (FIG. 32). Noted that the efficiency gap between inverted and regular devices have been decreased to ˜0.4% based on BHT@ZnO ETL. More excitingly, the device based on BHT@ZnO exhibited excellent stability under MPP tracking (FIG. 7d and FIG. 33). After continuously aging at MPP under light exposure for over 7724 h, the BHT@ZnO-based inverted OPV demonstrating an excellent lifetime with 81.5% PCE retention. To the best of our knowledge, it is the first for achieving such a long measured (rather than extrapolated) lifetime of OPV with a high certified PCE of 19%, including regular and inverted OPV devices (FIG. 7e and Table S6 in FIG. 40).

In summary, a multifunctional BHT@ZnO nanoparticle was designed and synthesized as an ETL for inverted OPV to simultaneously enhance efficiency and stability. BHT@ZnO exhibited decreased surface energy and reduced surface defects compared to unmodified ZnO. This modification resulted in a remarkable PCE of 19.47% in inverted OPVs. Notably, the inverted OPVs showed light soaking-free behavior and excellent stability under ISOS-D-1 (94.2% PCE retention after 8904 hours ambient storage without encapsulation) and ISOS-L-1 testing protocol (81.5% PCE retention after 7724 hours MPP tracking). Additionally, we discovered and elucidated the detailed degradation mechanism of the active layer systems, which involved the selective catalysis of PM6 and BTP-eC9 by superoxide and hydroxyl radicals, as well as the oxidation products of BDT-based donor materials upon radiation exposure. Furthermore, we demonstrated the ability of BHT@ZnO as an ETL to suppress the superoxide and hydroxyl radicals, thereby preventing degradation of the active layer molecules.

### 6. Photovoltaic Performance and Stability for GA@ZnO

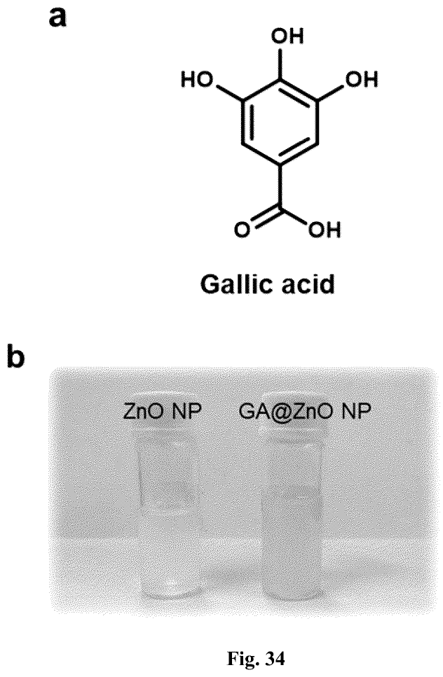

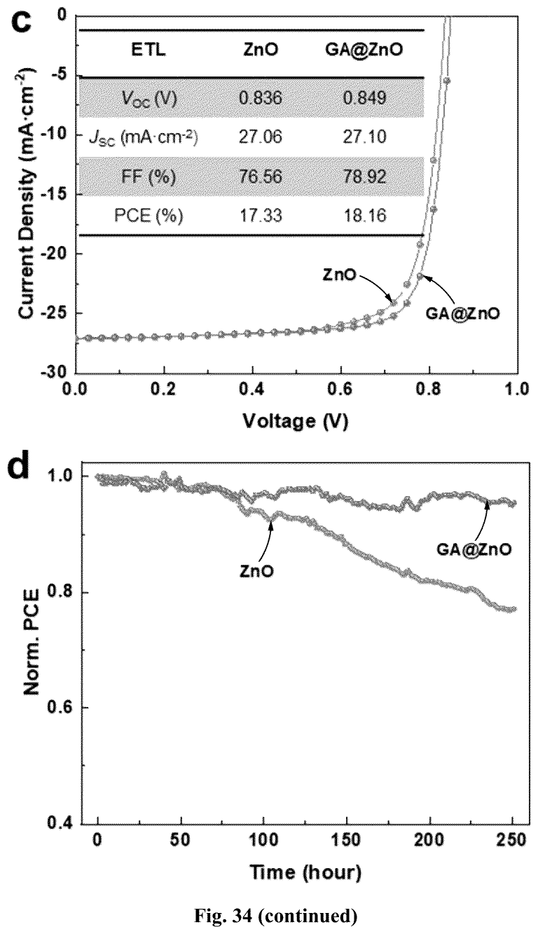

Another radical scavenger, gallic acid, was introduced for the modification of ZnO nanoparticles. The molecular structure of gallic acid is depicted in FIG. 34a. The polyphenol unit within gallic acid functions as a free radical scavenger, while the benzoic acid component serves as a defect passivator. Compared to ZnO nanoparticles, the gallic acid-capped ZnO (GA@ZnO) dispersion exhibits a faint pink color in methanol (FIG. 34b).

Preliminary evaluations were conducted for the photovoltaic performance and stability of devices utilizing GA@ZnO as the ETL. As illustrated in FIG. 34c, the device incorporating GA@ZnO ETL demonstrated a champion PCE of 18.16%, accompanied by improvements in V_{OC} and FF. Furthermore, the inverted device employing GA@ZnO exhibited enhanced stability, with approximately 95% PCE retention after approximately 250 hours of MPPT, surpassing the performance of devices utilizing ZnO NPs (FIG. 34d). These enhancements in efficiency and stability were attributed to our innovative strategy of multifunctional molecular engineering for defect passivation and free radical scavenging.

The present disclosure provides a deeper understanding of the degradation mechanism of active layer materials, offers a potential solution for ETL improvement, and has significant implications for the future design of active layer materials.

The disclosed experimental data was designed to establish the feasibility and reproducibility of the claimed process under representative conditions. The chosen materials and process parameters reflect the desired outcomes and are aligned with standard practices in the field. The focus of the current disclosure was to demonstrate the viability of the process under the specific conditions described. While the experimental data provided focuses on specific conditions, the process is not intended to be limited to these embodiments. The methodology described herein is adaptable to a range of conditions, and variations in the components could be explored to optimize the process for specific applications. The selection of the described parameters was based on their practical relevance and alignment with the objectives of this invention.

What is claimed is:

1. A metal oxide layer comprising a modified metal oxide nanoparticle, wherein the modified metal oxide nanoparticle comprises an organic acid metal salt on the surface of a metal oxide nanoparticle, and the organic acid metal salt has Formula 1: 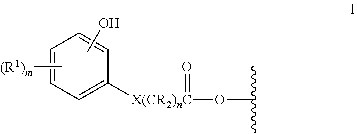
wherein
m is a whole number selected from 0-2;
n is a whole number selected from 0-12;
X is —O— or a bond;
R¹ for each instance is independently H, OH, alkyl or cycloalkyl;
R² for each instance is independently hydrogen or alkyl; or two instances of —CR²— taken together form a double bond; and [symbol]
represents a metal counterion.

2. The metal oxide layer of claim 1, wherein the metal oxide nanoparticle comprises an n-type metal oxide.

3. The metal oxide layer of claim 1, wherein the metal oxide nanoparticle comprises ZnO, SnO₂, TiO₂, In₂O₃, NiO, VOₓ, WO₃, or MoO₃.

4. The metal oxide layer of claim 1, wherein R¹ for each instance is independently branched alkyl.

5. The metal oxide layer of claim 1, wherein X is a bond, R¹ for each instance is branched alkyl, R² for each instance is hydrogen.

6. The metal oxide layer of claim 5, wherein m is a whole number selected from 1-2, and n is a whole number selected from 1-5.

7. The metal oxide layer of claim 1, wherein X is a bond, R¹ for each instance is OH, R² for each instance is hydrogen or alkyl

8. The metal oxide layer of claim 7, wherein m is a whole number selected from 1-2, and n is a whole number selected from 0-3.

9. The metal oxide layer of claim 5, wherein the metal oxide nanoparticle comprises ZnO.

10. The metal oxide layer of claim 1, wherein the modified metal oxide nanoparticle has an average particle size of 5-10 nm.

11. A method for producing the metal oxide layer of claim 1, the method comprising
providing a dispersion of modified metal oxide nanoparticle, and annealing the dispersion to form the metal oxide layer;
wherein the modified metal oxide nanoparticle is prepared by contacting a metal oxide nanoparticle with an organic acid thereby forming an organic acid metal salt distributed on the surface of the metal oxide nanoparticle; and the organic acid is represented by Formula 2: 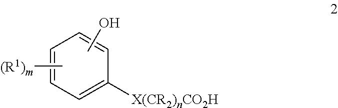
wherein
m is a whole number selected from 0-2;
n is a whole number selected from 0-12;
X is —O— or a bond;
R¹ for each instance is independently H, OH, alkyl or cycloalkyl; and
R² for each instance is independently hydrogen or alkyl; or two instances of —CR²— taken together form a double bond.

12. The method of claim 11, wherein X is a bond, R¹ for each instance is branched alkyl, R² for each instance is hydrogen.

13. The method of claim 11, wherein X is a bond, R¹ for each instance is OH, R² for each instance is hydrogen or alkyl.

14. The method of claim 11, wherein the organic acid is selected from the group consisting of:

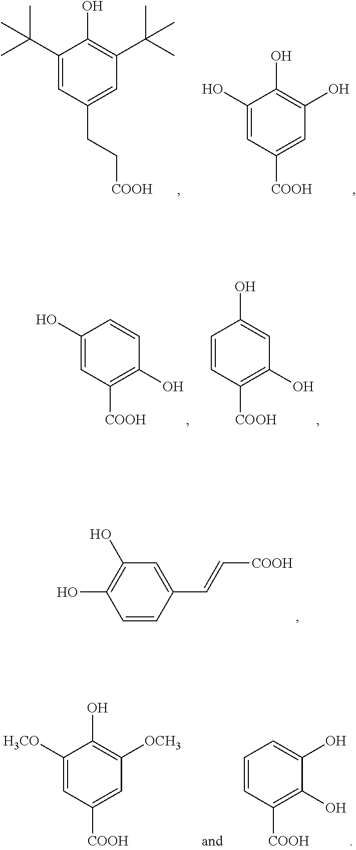

15. The method of claim 11, wherein the dispersion of modified metal oxide nanoparticle comprises a solvent, and the solvent comprises methanol, isopropanol, butanol, dimethyl formamide (DMF), dimethyl sulfoxide, N-methyl-2-pyrrolidone (NMP), N,N′-dimethylpropyleneurea (DMPU), or mixtures thereof.

16. The method of claim 11, wherein the metal oxide nanoparticle and the organic acid are contacted at a temperature of 10-50° C.

17. A coating structure comprising the metal oxide layer of claim 1 and an active layer coating on a surface of the metal oxide layer.

18. The coating structure of claim 17, wherein the active layer comprises poly[(2,6-(4,8-bis(5-(2-ethylhexyl-3-fluoro)thiophen-2-yl)-benzo[1,2-b:4,5-b′]dithiophene))-alt-(5,5-(1′,3′-di-2-thienyl-5′,7′-bis(2-ethylhexyl)benzo[1′,2′-c:4′,5‘-c’]dithiophene-4,8-dione)](PM6), 2,2′-[[12,13-Bis(2-butyloctyl)-12,13-dihydro-3,9-dinonylbisthieno[2″,3″:4′,5′]thieno[2′,3′:4,5]pyrrolo[3,2-e:2′,3′-g][2,1,3]benzothiadiazole-2,10-diyl]bis[methylidyne(5,6-chloro-3-oxo-1H-indene-2,1(3H)-diylidene)]]bis[propanedinitrile](BTP-eC9), 2,2′-((2Z,2′Z)-((12,13-bis(2-ethylhexyl)-3,9-(2-butyloctyl)-12,13-dihydro-[1,2,5]thiadiazolo[3,4-e]thieno[2″,3″:4′,5′]thieno[2′,3′:4,5]pyrrolo[3,2-g]thieno[2′,3′:4,5]thieno[3,2-b]indole-2,10-diyl)bis(methanylylidene))bis(5,6-difluoro-3-oxo-2,3-dihydro-1H-indene-2,1-diylidene))dimalononitrile(L8-BO), 2,2′-((2Z,2′Z)-((12,13-bis(2-ethylhexyl)-3,9-diundecyl-12,13-dihydro-[1,2,5]thiadiazolo[3,4-e]thieno[2″,3″:4′,5′]thieno[2′,3′:4,5]pyrrolo[3,2-g]thieno[2′,3′:4,5]thieno[3,2-b]indole-2,10-diyl)bis(methanylylidene))bis(5,6-difluoro-3-oxo-2,3-dihydro-1H-indene-2,1-diylidene))dimalononitrile(Y6), poly[(2,6-(4,8-bis(5-(2-ethylhexyl)-4-fluoro)thiophen-2-yl)-benzo[1,2-b:4,5-b′]dithiophene)-alt-5,5′-(5,8-bis(4-(2-butyloctyl)thiophen-2-yl)dithieno[3′,2′:3,4;2″,3″:5,6]benzo[1,2-c][1,2,5]thiadiazole)](D18), poly[(2,6-(4,8-bis(5-(2-ethylhexyl)-4-chlorothiophen-2-yl)-benzo[1,2-b:4,5-b′]dithiophene))-alt-5,5′-(5,8-bis(4-(2-butyloctyl)thiophen-2-yl)dithieno[3′,2′:3,4;2″,3″:5,6]benzo[1,2-c][1,2,5]thiadiazole)](D18-C1) poly[[12,13-bis(2-octyldodecyl)-12,13-dihydro-3,9-diundecylbisthieno[2″,3″:4′,5′]thieno[2′,3′:4,5]pyrrolo[3,2-e:2′,3′-g][2,1,3]benzothiadiazole-2,10-diyl]methylidyne[1-(dicyanomethylene)-1,3-dihydro-3-oxo-2H-inden-yl-2-ylidene]-2,5-thiophenediyl[1-(dicyanomethylene)-1,3-dihydro-3-oxo-2H-inden-yl-2-ylidene]methylidyne](PY-IT), poly[[4,8-bis[5-(2-ethylhexyl)-2-thienyl]benzo[1,2-b:4,5-b′]dithiophene-2,6-diyl]-2,5-thiophenediyl[5,7-bis(2-ethylhexyl)-4,8-dioxo-4H,8H-benzo[1,2-c:4,5-c′]dithiophene-1,3-diyl]](PBDBD-T) or mixtures thereof.

19. A organic photoelectric cell comprising the metal oxide layer of claim 1.

20. The organic photoelectric cell of claim 19, wherein the organic photoelectric cell has an inverted structure.
